SEQUENCE LISTING (54 sequences)

SEQ ID NO: 1 - protein, 607 aa, Plasma Factor XI
ECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAV

SEQ ID NO: 2 - protein, 553 aa, Platelet Factor XI
ECVTQLLKDTCFEGGDITTVFTPSAKYCQVVCTYHPRCLLFTFTAESPSEDPTRWFTCVLKDSVTETLPRVNRTAAISGYSFKQCSHQISNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAV

SEQ ID NO: 3 - protein, 13 aa, Artificial
  Synthetic
SRWPWSVFPDFPD

SEQ ID NO: 4 - protein, 13 aa, Artificial
  Synthetic
DVWDYVVFDDFPS

SEQ ID NO: 5 - protein, 14 aa, Artificial
  Synthetic
QRWVPYDDFPSLRS

SEQ ID NO: 6 - protein, 13 aa, Artificial
  Synthetic
RHFHVFPDFPFVH

SEQ ID NO: 7 - protein, 14 aa, Artificial
  Synthetic
HHFPPFSHFPDLPQ

SEQ ID NO: 8 - protein, 12 aa, Artificial
  Synthetic
RRLPLSRLPDFP

SEQ ID NO: 9 - protein, 12 aa, Artificial
  Synthetic
HPFFRGYPDFPD

SEQ ID NO: 10 - protein, 12 aa, Artificial
  Synthetic
HPWHLVYPDFPS

SEQ ID NO: 11 - protein, 12 aa, Artificial
  Synthetic
HDWLVRWPDFPS

SEQ ID NO: 12 - protein, 12 aa, Artificial
  Synthetic
SHFWRQWPDFSD

SEQ ID NO: 13 - protein, 13 aa, Artificial
  Synthetic
PQLRWHDFPDFGS

SEQ ID NO: 14 - protein, 14 aa, Artificial
  Synthetic
VVWRHWQDFDQFVV

SEQ ID NO: 15 - protein, 13 aa, Artificial
  Sytnthetic
VDWQWSRFDDFPS

SEQ ID NO: 16 - protein, 12 aa, Artificial
  Synthetic
HPWFDDFPHLFQ

SEQ ID NO: 17 - protein, 12 aa, Artificial
  Synthetic
YKWIHHDDFPLV

SEQ ID NO: 18 - protein, 12 aa, Artificial
  Synthetic
FDRKRVHPDFPH

SEQ ID NO: 19 - protein, 13 aa, Artificial
  Synthetic
DVWDYVVFDDFPS

SEQ ID NO: 20 - protein, 11 aa, Artificial
  Synthetic
QQPIQRFPDFP

SEQ ID NO: 21 - protein, 12 aa, Artificial
  Synthetic
QAIFTRFPDFPN

SEQ ID NO: 22 - protein, 12 aa, Artificial
  Synthetic
EWFPDFPEGSDG

SEQ ID NO: 23 - protein, 11 aa, Artificial
  Synthetic
HTHAFPDFPPH

SEQ ID NO: 24 - protein, 12 aa, Artificial
  Synthetic
LVKGFPDFPNHN

SEQ ID NO: 25 - protein, 12 aa, Artificial
  Synthetic
GPFPYAYEDFPE

SEQ ID NO: 26 - protein, 12 aa, Artificial
  Synthetic
FYLKTRYYDFPE

SEQ ID NO: 27 - protein, 12 aa, Artificial
  Synthetic
FQARHTIGDFPA

SEQ ID NO: 28 - protein, 12 aa, Artificial
  Synthetic
RIKDFPSDSNTV

SEQ ID NO: 29 - protein, 12 aa, Artificial
  Synthetic
IWESHKVIEDFP

SEQ ID NO: 30 - protein, 12 aa, Artificial
  Synthetic
QWFSVSRYQDFD

SEQ ID NO: 31 - protein, 12 aa, Artificial
  Synthetic
QKDFHWRILPDF

SEQ ID NO: 32 - protein, 12 aa, Artificial
  Synthetic
KIVKFPHTFPDL

SEQ ID NO: 33 - protein, 11 aa, Artificial
  Synthetic
HLYDFDLDNEY

SEQ ID NO: 34 - protein, 11 aa, Artificial
  Synthetci
KTILGDVDFDI

SEQ ID NO: 35 - protein, 11 aa, Artificial
  Synthetic
RQLHPFHHFHG

SEQ ID NO: 36 - protein, 10 aa, Artificial
  Synthetic
RSWLRYGYGH

SEQ ID NO: 37 - protein, 12 aa, Artificial
  Synthetic
FNWNNVDEYYDW

SEQ ID NO: 38 - protein, 12 aa, Artificial
  Synthetic
DQWDWEDYDEAW

SEQ ID NO: 39 - protein, 11 aa, Artificial
  Synthetic
YDIYDDYEIWA

SEQ ID NO: 40 - protein, 14 aa, Artificial
  Synthetic
YPKHIYADFPSTRL

SEQ ID NO: 41 - protein, 14 aa, Artificial
  Synthetic
YPRHIYPDFPTDTT

SEQ ID NO: 42 - protein, 14 aa, Artificial
  Synthetic
YLKHAWPDFPKLQQ

SEQ ID NO: 43 - protein, 14 aa, Artificial
  Synthetic
YVRHRFEDFPTALP

SEQ ID NO: 44 - protein, 14 aa, Artificial
  Synthetic
FPWHKYEDFPSPRT

SEQ ID NO: 45 - protein, 14 aa, Artificial
  Synthetic
QPAHRYPDFPRNNH

SEQ ID NO: 46 - protein, 14 aa, Artificial
  Synthetic
LPKTRFLDFPHVSF

SEQ ID NO: 47 - protein, 14 aa, Artificial
  Synthetic
LPPARYPDFPAAKK

SEQ ID NO: 48 - protein, 14 aa, Artificial
  Synthetic
IPKNRFSDFPDAQG

SEQ ID NO: 49 - protein, 14 aa, Artificial
  Synthetic
LPSFRFPDFPATKT

SEQ ID NO: 50 - protein, 14 aa, Artificial
  Synthetic
RVLNRYPDFPTTNQ

SEQ ID NO: 51 - protein, 14 aa, Artificial
  Synthetic
FFKKTYADFPTSQT

SEQ ID NO: 52 - protein, 14 aa, Artificial
  Synthetic
IFKKTYEDFPRFVY

SEQ ID NO: 53 - protein, 14 aa, Artificial
  Synthetic
VLHNKYDDFPRVKK

SEQ ID NO: 54 - protein, 14 aa, Artificial
  Synthetic
KVKHRFNDFPVWGN

(19) United States
(12) Patent Application Publication
(10) Pub. No.: US 2005/0181978 A1
(43) Pub. Date: Aug. 18, 2005

(54) Therapeutic use of factor XI

(76) Inventors: Rasmus Rojkjaer, Gentofte (DK); Dorthe Viuff, Frederikssund (DK); Soren Ostergaard, Bronshoj (DK); Simon Bjerregaard Jensen, Hillerod (DK); Jens Jacob Hansen, Jyllinge (DK)

Correspondence Address: NOVO NORDISK, INC.;PATENT DEPARTMENT, 100 COLLEGE ROAD WEST, PRINCETON, NJ, 08540, US

(21) Appl. No.: 10/993,906

(22) Filed: Nov. 19, 2004

Related U.S. Application Data
(60) Provisional application No. 60/523,849, filed on Nov. 20, 2003.
(60) Provisional application No. 60/593,000, filed on Jul. 30, 2004.

(30) Foreign Application Priority Data
Nov. 20, 2003 (DK) PA 2003 01721
Jul. 23, 2004 (DK) PA 2004 01141

(51) Int. Cl. A61K 38/36

(52) U.S. Cl. 514/2

(57) ABSTRACT

The present invention provides methods and compositions for treating bleeding episodes. The methods are carried out by administering to a patient in need thereof a preparation comprising a factor XI polypeptide, in an amount effective for such treatment. The methods of the invention result in one or more of: reduced clotting time; enhancement of hemostasis; increase in clot lysis time; increase in clot strength; and/or increase in overall clot quality (OCQ) in said patient.

### CROSS-REFERENCE TO RELATED APPLICATIONS

This application claims priority under 35 U.S.C. 119 of Danish application no. PA 2003 01721 filed Nov. 20, 2003, Danish application no. PA 2004 01141 filed Jul. 23, 2004, U.S. application Ser. No. 60/523,849 filed Nov. 20, 2003 and U.S. application Ser. No. 60/593,000 filed Jul. 30, 2004, the contents of each of which are fully incorporated herein by reference.

### FIELD OF THE INVENTION

The present invention relates to the therapeutic use of human Factor XI for the prevention and/or treatment of bleeding episodes, methods for the purification of factor XI and factor XI polypeptides from biological fluids, as well as pharmaceutical formulations.

### BACKGROUND OF THE INVENTION

Human Factor XI is a serine protease consisting of two identical subunits, each having a molecular mass of about 80 kDa. FXI circulates in plasma as a disulfide-linked homodimer having a molecular mass of ˜160 KDa. FXI is activated by cleavage of each monomer between Arg₃₆₉ and IIe₃₇₀ to form an amino-terminal heavy chain of 50 kDa and a carboxy-terminal light chain of 35 kDa, which are disulfide-linked. The protein is encoded by a 23 kb gene located on chromosome 4 (4q35) 15 exons and 14 introns coding for a mRNA consisting of 2,097 nucleotides, which in turn encodes an amino-terminal signal (leader) peptide of 18 amino acids and the 607 amino acids present in each monomer of the mature protein. Exons III-X encode four tandem repeats sequences (Apple domains) homologous to similar domains found in human plasma PK (58% identity). Exons XI-XV encode the typical trypsin-like catalytic domain, which is activated by proteolytic cleavage of the zymogen at an internal Arg 369-IIe 370 bond to yield a heavy chain containing four Apple domains (369 amino acids) and the light chain or catalytic domain (238 amino acids).

One mechanism for initiation of coagulation is via exposure to the circulation of tissue factor (TF) at sites of injury, followed in succession by (i) binding of plasma Factor VII (FVII) to TF and its proteolytic conversion to activated Factor VII (FVIIa); (ii) binding of Factor X to the TF-FVIIa complex and its proteolytic conversion to activated Factor X (FXa); (iii) proteolytic conversion by FXa of prothrombin to thrombin; and (iv) the generation of a complex between tissue factor pathway inhibitor (TFPI) and FXa, followed by binding of the TFPI:FXa complex to TF-FVIIa, which attenuates FXa activation of thrombin and limits the flux of thrombin generated via the TF pathway. The relatively small amount of thrombin produced during this phase results in the activation of FXI to FXIa (which activates Factor IX to FIXa) and the activation of Factor V on the surface of platelets and the further activation of Factor X. These events further promote the formation of sufficient amounts of thrombin (the so-called “thrombin burst”) to convert fibrinogen into fibrin, thereby stabilizing an initial platelet plug and resulting in appropriate hemostasis.

Dimeric FXI circulates in plasma as a zymogen in a non-covalent complex with the cofactor high molecular weight kininogen (HK) that promotes the binding of FXI to negatively charged surfaces and its activation by its cognate proteases, FXIIa, FXIa, and thrombin. The HK binding site to FXI involves multiple Apple domains (A1, A2, A4), with the A2 domain being the most important. Complex formation with HK in the presence of Zn²⁺ ions has been shown to promote the binding of FXI to activated platelets. The interaction of FXI with the surface of activated platelets has been shown to be mediated via residues Ser₂₄₈-Val₂₇₁ within the A3 domain of FXI; residues Ser₂₄₈, Arg₂₅₀, Lys₂₅₅, Phe₂₆₀ and GIn₂₆₃ have also been implicated in this interaction. The A3 domain of FXI also contains a heparin binding site within residues Thr₂₄₉-Phe₂₆₀ and residues Lys₂₅₂ and Lys₂₅₃ have been implicated in the binding to platelets. Although FXI and HK circulate in plasma in a non-covalent complex, and HK has been shown to bind to the surface of activated platelets, the interaction of FXI with the platelet surface apparently does not require binding of HK-FXI complex. Instead, it appears that the FXI dimer binds directly to a high-affinity, specific site on activated platelets (approx. 1500 sites/platelet; K_{d} at approx 10 nM). The isolated recombinant A3 domain of FXI binds to the same number of sites on activated platelets and with the same affinity as the FXI dimer.

The activated enzyme, FXIa, has also been shown to bind to high-affinity, saturable sites on activated platelets (Kd at approx 800 pM; 500 sites/platelet) and can activate FIX at rates similar to those observed in solution. The substrate FIX binding site in FXI involves both a subdomain (Ala₁₃₄-Leu₁₇₂) in the A2 domain and two subdomains (Ile₁₈₄-Val₁₉₂ and Ser₂₅₉-Ser₂₆₅) within the A3 domain. Binding to the platelet surface is mediated by the glycoprotein Ib-V-IX complex utilizing one polypeptide chain of the FXI dimer, thereby presenting the other monomer as a substrate binding site for FIX. It is likely that FIXa generation serves to localize FIXa-catalyzed FX activation to the platelet surface which also promotes prothrombin activation by FXa.

In addition to forming membrane associated complexes leading to the local explosive generation of thrombin on the platelet surface, FXIa is also subject to regulation by a variety of plasma and platelet protease inhibitors whose functional activity appears to depend on whether FXIa is bound to the platelet surface or whether it is free in solution. Thus, a number of serine protease inhibitors including α-1-protease inhibitor, antithrombin III, C1 inhibitor, α-2-antiplasmin, plasminogen activator inhibitor 1 (PAI-1), and protein C inhibitor have all been shown to inactivate FXIa in the plasma compartment. However, within the environment of activated platelets, it seems likely that the most physiologically relevant inhibitor of FXIa is protease nexin II (PNII), which is found in very low concentration in plasma but is secreted from platelet α-granules (1-1.5 nM PNII released per 10⁸ platelets) suggesting a plasma concentration at 3-5 nM under normal physiological conditions. PNII is a potent inhibitor of FXIa with a Ki of 300-500 pM that is significantly enhanced in the presence of heparin. Binding of FXI to the platelet surface in the presence of HK and Zn²⁺ ions or in the presence of prothrombin and Ca²⁺ is protected from inactivation by both PNII and α-i-protease inhibitor showing that FXIa activity generated on the surface of platelet is localized to the haemostatic thrombus whereas the site of regulation of FXIa by PNII and other protease inhibitors occurs in solution. It is also possible that endothelial cells, which contain heparan sulphate glycosaminoglycans, might promote the assembly of FXIa/PNII complexes thereby potentiating the inhibition of FXIa on the endothelium.

The participation of FXI in thrombin generation on the surface of a clot is also thought to play a role in inhibiting fibrinolysis via thrombin-activatable fibrinolysis inhibitor (TAFI), which proteolytically removes carboxy-terminal lysine residues from fibrin that play a role in plasminogen binding and activation. An intact FXI feedback loop is believed to be necessary to generate sufficient thrombin for significant TAFI activation.

Notably, platelets and megakaryocytes apparently synthesize a second form of FXI, designated platelet-derived FXI (pd-FXI), which differs from the circulating form in lacking Exon V, which is the first exon of the two exons encoding the second Apple domain, and in vitro studies have shown that the preferred substrate for platelet factor XIa may be plasma FXI and not FIX. Platelet FXI (Mr 220 KDa) has been found to be associated with the platelet plasma membrane. Platelets contain about 300 molecules of pd-FXI/cell.

FXI deficiency is an autosomal recessive syndrome characterized by a variable tendency to bleed. Even if severe, the deficiency may be clinically asymptomatic until the patient is challenged by surgical trauma; however, in some cases bleeding can occur regardless of the severity of the deficiency. Optimal management of patients with FXI deficiency requires attention to a number of features in addition to the FXI level. First, it is important to evaluate the bleeding tendency in an individual with partial deficiency and whether additional factors are making a significant contribution. Such assessment should include measurement of FVIIIC and von Willebrand factor levels, the bleeding time and platelet aggregation. Fresh frozen plasma has been used to treat the first known cases of FXI deficiency and was the main treatment until the development of FXI concentrate. The main disadvantages of plasma are the large volumes required, allergic reactions and the potential for transmission of infectious agents. In addition, there have been reported a rather variable FXI content in this product. Two FXI concentrates are currently available. The FXI concentrate from Bio Products Laboratory (BPL) (England) is formulated with a high concentration of antithrombin (mean 102 iu/ml) and heparin (10 u/ml) which is thought to protect against any residual FXIa. A second FXI concentrate is produced by Hemoleven (France) and the product is formulated with 3-5 u/ml heparin, 2-3 iu/ml of antithrombin and C1 inhibitor. Furthermore, it has been reported that it is possible to pasteurize pooled fresh frozen plasma with preservation of 75-95% activity of FXI. Patients with mild FXI deficiency are generally treated with fresh frozen plasma, though patients with severe FXI deficiency may be treated with a FXI concentrate.

Patients (including those not suffering from congenital FXI deficiency) who bleed excessively in association with surgery or major trauma and need blood transfusions develop more complications than those who do not experience any bleeding. Even moderate bleedings requiring the administration of human blood or blood products (such as, e.g., platelets, leukocytes, plasma-derived concentrates for the treatment of coagulation defects, etc.) may lead to complications associated with the risk of transferring human viruses (hepatitis, HIV, parvovirus, and other, presently unknown viruses). Extensive bleedings requiring massive blood transfusions may lead to the development of multiple organ failure including impaired lung and kidney function. Once a subject has developed these serious complications a cascade of events involving a number of cytokines and inflammatory reactions is started making any treatment extremely difficult or, often, unsuccessful. Therefore a major goal in surgery as well as in the treatment of major tissue damage is to avoid or minimise the bleeding. To avoid or minimise such bleeding, it is of importance to ensure the formation of stable and solid haemostatic plugs that are not easily dissolved by fibrinolytic enzymes. Furthermore, it is of importance to ensure quick and effective formation of such plugs or clots.

WO2003007983 discloses the use of a combination of factor VIIa and FXI for treatment of bleeding episodes.

Thus, there is a need in the art for improved hemostatic treatment modalities that result in the rapid, controlled formation of stable fibrin clots.

### SUMMARY OF THE INVENTION

The present invention provides methods and compositions for treating bleeding episodes. The methods are carried out by administering to a patient in need thereof a preparation comprising an isolated factor XI (FXI) polypeptide, in an amount effective for such treatment. The methods of the invention result in one or more of: reduced clotting time; enhancement of hemostasis; increase in clot lysis time; increase in clot strength; and/or increase in overall clot quality (OCQ) in said patient. In some embodiments, following administration of a FXI polypeptide, the patient exhibits an effective FXI plasma concentration of at least about 5 nM, 10 nM, or 30 nM.

In some embodiments, the FXI polypeptide comprises the sequence of SEQ ID NO:1, or a fragment thereof that retains at least one FXI-associated biological activity. In some embodiments, the FXI polypeptide comprises the sequence of SEQ ID NO:2, or a fragment thereof that retains at least one FXI-associated biological activity. In some embodiments, the FXI polypeptide comprises a chemically modified derivative of SEQ ID NO:1 or SEQ ID NO:2, or a variant of either SEQ ID NO:1 or SEQ ID NO:2 containing one or more amino acid sequence alterations. In some embodiments, the FXI polypeptide has the sequence of SEQ ID NO:1. In some embodiments, the FXI polypeptide has the sequence of SEQ ID NO:2. In some embodiments, the FXI polypeptide is recombinantly produced.

In some embodiments, the patient does not suffer from a congenital FXI deficiency. In some embodiments, the bleeding episodes are secondary to surgery, a dental procedure, trauma, or hemodilution.

The invention also provides methods and compositions for preventing bleeding episodes. The methods are carried out by administering to a patient in need thereof a preparation comprising a FXI polypeptide, in an amount effective to prevent bleeding.

In some embodiments, the methods of the invention further comprise, prior to administration of a FXI polypeptide: (a) obtaining a sample of blood from said patient; (b) determining at least one of: FXI concentration, ratio of FXIa:FXI, or amount of exogenous FXI necessary to restore coagulation; and (c) based on the results of step (b), determining said amount of FXI effective for treatment.

In one embodiment the methods of the invention does not comprise administration of a Factor VII/Factor VIIa coagulation agent.

As used herein, a Factor VII/Factor VIIa coagulation agent is a Factor VII polypeptide or a Factor VII-related polypeptide as described in WO2003007983.

The invention also provides methods and compositions for treating bleeding episodes in which a patient is administered (i) a first amount of a preparation comprising a FXI polypeptide and (ii) a second amount of a preparation comprising a non-Factor VII/Factor VIIa coagulation agent, under conditions in which the first and second amounts in combination are effective for such treatment. Non-limiting examples of non-Factor VII/Factor VIIa coagulation agents include: Factor XII; tissue factor pathway inhibitor (TFPI) inhibitor; Factor IX; thrombin activatable fibrinolysis inhibitor (TAFI); plasminogen activator inhibitor-1 (PAI-1); Factor VI; protein C inhibitor; protein S inhibitor; tissue plasminogen activator (tPA) inhibitor; prothrombin, Factor VIII, fibrinogen, and Factor X.

The invention also provides pharmaceutical formulations comprising (i) an isolated recombinant FXI polypeptide and (ii) a pharmaceutically acceptable carrier or excipient.

The invention further provides methods for purifying a factor XI polypeptide from a biological material, the method comprising subjecting the material to sequential chromatography on an cation-exchange chromatographic material, a hydrophobic interaction chromatographic material, and a Hydroxyapatite chromatographic material.

### BRIEF DESCRIPTION OF THE DRAWINGS

FIG. 1 is a graphic representation of the effect of increasing amounts of FXI on overall clot quality in blood obtained from patients before and after cardiac surgery.

FIG. 2 is a graphic representation of the effect of increasing amounts of FXI on overall clot quality in blood obtained from normal subjects.

FIG. 3 is a graphic representation of the biological activity of different FXI formulations after storage at 50° C. for 96 days.

FIG. 4 is the preparative chromatogram of factor XI polypeptide-containing fractions from first cation-exchange chromatography using Obelix ST CIEX (cat no 11-0010) as described in example 7.

FIG. 5 is the preparative chromatogram of factor XI polypeptide-containing fractions from Hydrophobic interaction chromatography using Butyl Sepharose High Performance High Substitution (cat no 17-3100) as described in example 8.

FIG. 6 is the preparative chromatogram of factor XI polypeptide-containing fractions from Hydroxyapatite chromatography using CHT Hydroxyapatite Type I BioRad cat no 157-0020) as described in example 9.

### DETAILED DESCRIPTION OF THE INVENTION

The present invention is based on the surprising finding that exogenously administered Factor XI (FXI) can be effective as a general hemostatic agent in human blood without the administration of Factor VII/Factor VIIa coagulation agents. The therapeutic use of FXI according to the invention may provide one or more of: a shortened clotting time, a firmer clot, and an increased resistance of the formed clots to fibrinolysis.

The present invention provides methods and compositions useful in the therapeutic use of FXI in human patients for treating or preventing bleeding episodes, for enhancing hemostasis, for increasing clot lysis time, and/or for increasing clot strength. The methods are carried out by administering to the patient an effective amount of Factor XI for achieving one or more of these desired therapeutic goals. The compositions include, without limitation, pharmaceutical formulations for the therapeutic use of FXI that comprise isolated recombinant FXI.

In one series of embodiments, the present invention relates to administration of FXI to normal human patients. As used herein, a “normal” human is one who does not suffer from a congenital deficiency in Factor XI (i.e., Hemophilia C, see, Seligsohn (1993), Thromb. Haemost. 70:68-71); normal humans include, without limitation, patients exhibiting thrombocytopenia (lowered count or activity of platelets), patients contemplating or undergoing a surgical or dental procedure, and patients who have been subjected to trauma or organ damage and who, as a consequence, may exhibit lowered platelet counts and/or lowered levels of fibrinogen, FVIII, and/or other coagulation proteins. Normal human patients, for example, encompass patients experiencing a transient decrease in the plasma levels of FXI (or any other coagulation-related protein or factor) due to bleeding, trauma, chemotherapy, liver disease, hemodilution (such as, e.g., may result from the infusion of plasma expanders or salt solutions to maintain blood volume or prevent shock), or any other circumstances not directly related to a congenital defect in a FXI gene.

In another series of embodiments, the present invention relates to administration of FXI to patients suffering from Hemophilia A or B.

In another series of embodiments, the present invention relates to administration of isolated and/or recombinant FXI to human patients suffering from a congenital FXI deficiency.

In practicing the present invention, any FXI polypeptide may be used that is effective in preventing or treating bleeding. This includes FXI polypeptides derived from blood or plasma or from platelets or those produced by recombinant means in any suitable host organism or cell. Also encompassed are FXI polypeptides in their uncleaved (zymogen) form, as well as those that have been proteolytically processed to yield their respective bioactive forms (designated FXIa).

As used herein, FXI polypeptides encompass, without limitation, FXI as well as FXI-related polypeptides. The term “FXI” is intended to encompass, without limitation, polypeptides having the amino acid sequence of wild-type human plasma FXI, as described, e.g., in Fujikawa et al., Biochem. 25:2417 (1986), as well as wild-type FXI derived from other species, such as, e.g., bovine, porcine, canine, murine, rabbit, and salmon FXI. In general, it is preferred to use FXI proteins syngeneic with the subject, in order to reduce the risk of inducing an immune response. Preparation and characterization of non-human FXI has been described, e.g., by Gailani (1997), Blood 90:1055. The present invention also encompasses the use of such factor XI proteins within veterinary procedures.

In some embodiments, the FXI polypeptide is wild-type human plasma FXI (SEQ ID NO:1). In other embodiments, the FXI is platelet-derived FXI (pd-FXI) (SEQ ID NO:2), as described, e.g., in Hsu et al. (1998), J. Biol. Chem. 273:13787-93.

FXI polypeptides further encompass natural allelic variations of FXI that may exist and differ from one individual to another. Also, the degree and location of glycosylation or other post-translational modifications may vary in some circumstances, depending on the source of FXI-encoding nucleic acid, the host cells in which the FXI is produced, and the conditions in which the FXI-producing cells are maintained.

FXI-related polypeptides include, without limitation, FXI polypeptides that have either been chemically modified relative to human FXI (i.e., FXI derivatives) and/or contain one or more amino acid sequence alterations relative to human FXI (i.e., FXI variants). Such FXI-related polypeptides may exhibit an alteration in one or more aspects of biological activity relative to human FXI, including, without limitation, altered stability, altered phospholipid binding, altered specific enzymatic activity, altered immunogenicity, altered bioavailability, altered binding to one or more FXI binding partners, altered binding to FXI inhibitors, and the like. FXI-related polypeptides encompass such polypeptides in their uncleaved (zymogen) form, as well as those that have been proteolytically processed to yield their respective bioactive forms, which may be designated “FXIa-related polypeptides” or “activated FXI-related polypeptides”.

Non-limiting examples of FXI derivatives include: wild-type FXI or FXI variants that have been modified by phosphorylation, sulfation, PEGylation, or by the action of one or more glycosyltransferases and/or glycosidases, whether in vivo or in vitro (see, e.g., Ekdahl et al. (1999), Thromb. Haemost. 82:1283-8).

Non-limiting examples of FXI variants include: FXI in which one or more N-linked or O-linked glycosylation consensus sites have been modified, single-chain FXI (i.e., FXI in which the monomer polypeptides are not subject to intrachain proteolytic cleavage as in the wild-type), and cysteine variants in which one or more cysteine residues are eliminated or relocated, including, but not limited to, alterations that change the disulfide bonding pattern of the monomer or dimer. In one embodiment, Cys₁₁ (which is not believed to participate in inter- or intramolecular disulfide bonding) is eliminated or substituted.

In one series of embodiments, the FXI variant has decreased half-life in plasma relative to wild-type human FXI. In one embodiment the FXI variant has a half-life lower than 50 hours. In one embodiment the FXI variant has a half-life lower than 24 hours. In one embodiment the FXI variant has a half-life lower than 12 hours. In one embodiment the FXI variant has a half-life lower than 6 hours. In one embodiment the FXI variant has a half-life lower than 3 hours.

In one series of embodiments, FXI variants are polypeptides in which N-linked glycosylation at one or more sites has been disrupted by modified of the cognate N-linked glycosylation consensus sites, such as, e.g., by independent substitution with any amino acid of N90, N126, N353, N450, N491, or combinations of any of the foregoing. Non-limiting examples of such variants include FXI-N72Q; FXI-N108Q; FXI-N335Q, FXI-N432Q, FXI-N473Q; FXI-N72Q/N108Q; FXI-N72Q/N108Q/N335Q; FXI-N72Q/N108Q/N335Q/N432Q; FXI-N72Q/N108Q/N335Q/N432Q/N473Q; FXI-N72Q/N432Q; FXI-N72Q/N473Q; FXI-N108Q/N432Q FXI-N108Q/N473Q; and FXI-N432Q/N473Q. Disruption of N-linked glycosylation at one or more of the sites may also be achieved, e.g., by: (i) independent deletion of any of residues 72-74, 108-110, 335-337, 432-434, and 473-475 (i.e., one or more residues at each site may be deleted and not substituted with any another amino acid) (ii) independent substitution of the N+2 residue (such as, e.g., substituting T74 to any residue other than S, substitution of S110 to any residue other than T, substitution of S337 to any residue other than S, substitution of S434 to any residue other than T, substitution of T475 to any residue other than T; (iii) substitution of the N+1 residues with a glycosylation-disrupting amino acid (exemplified by, but not limited to, proline (P). It will be understood that any combination of the above means may be used to independently disrupt glycosylation at different sites within the FXI polypeptide.

Also encompassed by the invention are chimeric or fusion polypeptides between all or part of the FXI sequence and other heterologous peptide sequences. For example, one or more of the four Apple domains may be substituted by similar apple domains from other polypeptides (see, e.g., Gailani et al.(1999) Blood 94:621 a) or one or more of the Apple domains may be deleted in its entirety. In another embodiment, a binding site for LDL Receptor-associated protein (LRP) (such as, e.g., a peptide comprising residues Phe₃₄₂-Asn₃₄₆ of Factor IXa, which has been shown to contribute to the interaction with LRP, Rohlena et al. (2003), J. Biol. Chem. 278:9394) is attached to the sequence of a FXI polypeptide to modify its pharmacokinetic properties.

The dimeric nature of FXI in its active form (and the asymmetric function of the two monomers in, e.g., platelet binding and FIX activation) also enables preparations for use in the present invention that comprise FXI heterodimers, i.e., combinations of two non-identical FXI (or FXI-related) monomer polypeptides. The only requirement is that the heterodimer exhibit one or more beneficial aspects of FXI bioactivity.

FXI polypeptides for use in the present invention include, without limitation, polypeptides exhibiting substantially the same or improved biological activity relative to wild-type human FXI, as well as polypeptides in which the FXI biological activity has been substantially modified or reduced relative to the activity of wild-type human FXI.

In practicing the present invention, useful compositions comprising FXIa or FXIa-related polypeptides, including variants, encompass those that exhibit at least about 10%, at least about 20%, at least about 30%, at least about 40%, at least about 50%, at least about 60%, at least about 70%, at least about 80%, at least about 90%, at least about 100%, at least about 110%, at least about 120%, or at least about 130%, of the specific activity of compositions comprising solely wild-type FXI, when the wild-type FXI equivalent is one that has been obtained from the same source or produced in the same cell type, and when the activity comparison is made by parallel testing in an identical FXI activity assay. As used herein, the terms “activity” and “specific activity” apply, individually or in aggregate, to any aspect or aspects of FXI bioactivity.

In some embodiments, the ratio between the specific proteolytic activity of a FXI-related polypeptide and the proteolytic activity of wild-type human FXI is at least about 1.25 when tested a FXI amidolytic assay; in other embodiments, the ratio is at least about 2.0; in further embodiments, the ratio is at least about 4.0.

FXI Biological Activity

In practicing the present invention, one or more different aspects of FXI bioactivity may be quantified and used, e.g., in (i) selection of: appropriate FXI compositions for therapeutic administration, formulations, methods for FXI production or purification, and the like; and/or (ii) assessment of the efficacy of different therapeutic modalities. It will be understood that “specific activity” of FXIa for any of these aspects of bioactivity is expressed as units of activity per unit mass of FXIa polypeptides. These aspects include the following:

I. Proteolytic Activity:

(a) Amidolytic activity may be quantified in vitro using a suitable chromogenic substrate, such as, e.g., S2355 (Chromogenix), as described in Ekdahl et al. (1999), Thromb. Haemost. 82:1283-8. The measured activity is compared with a standard FXIa preparation having a defined specific activity (Enzyme Research Laboratories) and values are expressed as AU of FXIa activity.

(b) FXI activation activity may be quantified directly in vitro by measuring the proteolytic conversion of factor IX to IXa as described for example, in Gailani et al. (2001), Blood 97: 3117-3122.

II. Binding Activities and Inhibitors

Wild-type human FXI has a number of binding partners, including prekallikrein (PK), high-molecular weight kininogen (HK), thrombin/prothrombin, Factor IX (FIX), and the platelet-associated FXI receptor designated GP1b-V-IX. In practicing the present invention, any conventional binding assay may be used to quantify the affinity of FXI polypeptides for any of these (or other) binding partners. Such binding assays include, but are not limited to, competition binding assays in which either binding partner is labelled.

Also encompassed by FXI polypeptide binding partners are FXI active site inhibitors, including, without limitation, antithrombin III, C1 inhibitor, α2 antitrypsin, PAI-1, protein C inhibitor, and protease nexin II (PNII). The affinity of these compounds for FXI polypeptides may be quantified by use of conventional binding assays; alternatively, the inhibitory activity of such compounds for the proteolytic activity of particular FXI polypeptide preparations may be measuring using an amidolytic or FIX-activation assay.

III. Clotting Parameters:

Clotting time, clot lysis time, and clot strength are clinical parameters used for assaying the status of patient's haemostatic system. Blood samples are drawn from the patient at suitable intervals after administration of a FXI polypeptide and one or more of these parameters are assayed. Alternatively, a FXI polypeptide or preparation may be used for in vitro/ex vivo treatment of blood that has been drawn from a human subject.

Clotting time may be assayed by means of standard PT or aPTT assays.

Clot lysis time and clot strength may be measured by thromboelastograpy as described by, e.g., Vig et al. (2001) Blood coagulation & fibrinolysis, Vol. 12 (7) pp. 555-561. and Sorensen (2003) Throm Haemost 1:551-558. Alternatively, clot strength may be assayed as described by Carr et al, (1991), Am. J. Med. Sci. 302: 13-8.

One parameter that reflects the clotting activity of FXI as measured by thromboelastography is the “overall clot quality” (OCQ). Once clot formation has been initiated (t=0), measurement of the clot strength as a function of time reveals a maximum velocity (max vel) of clot formation as well as the time required to reach the maximum velocity (t_{max vel}). Subsequently, addition of tissue plasminogen activator (tPA) allows measurement of fibrinolysis and derivation of the time required to reach the maximum velocity of fibrinolysis (t_{min vel}) OCQ is calculated as:
(Max vel/t_{max vel})×(t_{min vel}−t_{max vel}).
IV. Pharmacokinetic Parameters

Wild-type human FXI is believed to have a half-life in plasma of approximately 50 hours, which is mediated at least in part to its interaction with HK. In practicing the present invention, FXI polypeptides, which exhibit pharmacokinetic properties that differ from native FXI, may be used. Non-limiting examples include FXI polypeptides that have been treated with sialidase to remove one or more terminal sialic acid residues from FXI-associated oligosaccharides, FXI polypeptides that have been modified by PEGylation, and FXI polypeptides exhibiting an altered interaction with HK. In practicing the present invention, pharmacokinetic properties may be calculated using, e.g., WinNonlin Professional Version 3.1 (Pharsight Inc., Mountain View, Calif., USA). Calculations are performed using mean concentration values at each time point, if more than one value was present.

The following pharmacokinetic parameters may be calculated: AUC, AUC_{%Extrap}, Cₘₐₓ, tₘₐₓ, λ_{z}, t_{1/2}, CL, and V_{z} using the following formulas:
AUC Area under the plasma concentration-time curve from time 0 to infinity. Calculated using the linear/log trapezoidal rule with extrapolation to infinity.
The linear trapezoidal rule is used from time 0 to tₘₐₓ:

$$AUC ( 0 - {t}_{max} ) = ( {\sum }_{i = 1}^{n - 1} \frac{C ( i ) + C ( i + 1 )}{2} \cdot ( t ( i + 1 ) - t ( i ) ) )$$
The log trapezoidal rule is used from time tₘₐₓ to the last time point t:

$$AUC ( {t}_{max} - t ) = ( {\sum }_{i = 1}^{n - 1} \frac{C ( i ) - C ( i + 1 )}{ln ( \frac{C ( i )}{C ( i + 1 )} )} \cdot ( t ( i + 1 ) - t ( i ) ) )$$
Extrapolation to infinity is performed using:

$$AUC ( t - ∞ ) = \frac{C ( t )}{{λ}_{z}}$$
AUC_{%Extrap} Percentage of AUC that is due to extrapolation from the last concentration to infinity:

$${AUC}_{\% Extrap} = \frac{AUC ( t - ∞ )}{AUC} \cdot 100 \%$$
Cₘₐₓ Maximum plasma concentration back extrapolated to time zero
CL Total body clearance

$$CL = \frac{Dose}{AUC }$$
tₘₐₓ Time at which maximum plasma concentration is observed.
t_{1/2} Half-life:

$${t}_{1 / 2} = \frac{ln 2}{{λ}_{z}}$$
λ_{z} Terminal rate constant. Calculated by log-linear regression of (mean) concentrations versus time
V_{z} Volume of distribution based on the terminal phase:

$${V}_{z} = \frac{Dose}{AUC \cdot {λ}_{z}}$$

Production and Purification of FXI:

FXI polypeptides for use in the present invention may be prepared from plasma or from recombinant sources using any suitable method known in the art. As used herein, the term “isolated” refers to FXI polypeptides that have been separated from the cell in which they were synthesized or the medium in which they are found in nature (e.g., plasma or blood).

Separation of polypeptides from their cell of origin may be achieved by any method known in the art, including, without limitation, removal of cell culture medium containing the desired product from an adherent cell culture; centrifugation or filtration to remove non-adherent cells; and the like. Optionally, FXI polypeptides may be further purified. Purification may be achieved using any method known in the art, including, without limitation, affinity chromatography, such as, e.g., on an anti-FXI antibody column or a peptide affinity column (non-limiting examples of which include Heparin, Blue, Red, L-arginine, Benzamidine peptide, other dyes, or RP-chromatography); hydrophobic interaction chromatography; ion-exchange chromatography; size exclusion chromatography; electrophoretic procedures (e.g., preparative isoelectric focusing (IEF), differential solubility (e.g., any precipitation or crystallization using, e.g., salt, pH, ammonium sulphate, or other additives), or extraction and the like, as described in more detail above. Following purification, the preparation preferably contains less than about 10% by weight, more preferably less than about 5% and most preferably less than about 1%, of non-FXI polypeptides derived from the host cell.

Purification of FXI from plasma may also be achieved by known methods, including, without limitation, those disclosed by Koide et al. (1977), Biochem. 16: 2279 and Bouma et al. (1977), J.Biol.Chem. 252:6432, incorporated herein by reference. Methods for preparing recombinant FXI are known in the art. See, for example, Kemball-Cook et al. (1994), Gene 139:275, Fujikawa et al. (1986), Biochem. 25:2417, and Meijers et al.(1992), Blood 79:1435, which are incorporated herein by reference in their entirety. FXIa is also commercially available from Enzyme Research Laboratories, South Bend, Ind.

The present invention further concerns a method for purifying a FXI polypeptide, such as recombinant FXI, from other biological material, the method comprising subjecting the material to chromatography on a cation-exchange chromatographic material.

The present invention further concerns a method for purifying a FXI polypeptide, such as recombinant FXI, from other biological material, the method comprising subjecting the material to chromatography on a a hydrophobic interaction chromatographic material.

The present invention further concerns a method for purifying a FXI polypeptide, such as recombinant FXI, from other biological material, the method comprising subjecting the material to chromatography on a hydroxyapatite chromatographic material.

The present invention further concerns a method for purifying a FXI polypeptide, such as recombinant FXI, from other biological material, the method comprising subjecting the material to sequential chromatography on a cation-exchange chromatographic material, a hydrophobic interaction chromatographic material and Hydroxyapatite chromatographic material. It is to be understood that a sequential chromatography is performed in the order as described.

The term “Hydroxyapatite chromatographic material” as used herein means any Hydroxyapatite chromatographic material known in the art which is capable of binding a FXI polypeptides, such as a Hydroxyapatite matrix.

The term “cation-exchange chromatographic material” as used herein means any cation-exchange chromatographic material known in the art which is capable of binding a FXI polypeptides, such as a cation-exchange matrix.

The term “hydrophobic interaction chromatographic material” as used herein means any hydrophobic interaction chromatographic material known in the art which is capable of binding a FXI polypeptides, such as a hydrophobic interaction matrix.

In one embodiment, the present invention concerns a method for purifying a FXI polypeptide from a biological material, the method comprising the step of: subjecting a biological material comprising a FXI polypeptide to chromatography on a first cation-exchange chromatographic material, said chromatography comprising:
(i) applying said biological material to said first cation-exchange chromatographic material;
(ii) eluting unbound material from the first cation-exchange chromatographic material with a buffer A, which buffer A is suitable for eluting material not bound to the first cation-exchange chromatographic material; and
(iii) eluting unbound material from the first cation-exchange chromatographic material with a buffer A′, which buffer A′ is suitable for eluting material not bound to the first cation-exchange chromatographic material; and
(iv) eluting said FXI polypeptide from the first cation-exchange chromatographic material by elution with buffer A′, which buffer A′ is suitable for eluting said FXI polypeptide from said first cation-exchange chromatographic material.

In one embodiment, the present invention concerns a method for purifying a FXI polypeptide from a biological material, the method comprising the steps of: subjecting the eluate from step (iv) or a fluid prepared by use of the eluate from step (iv) to chromatography using a hydrophobic interaction chromatographic material, said chromatography comprising:
(v) applying the eluate from step (iv) or a fluid prepared by use of the eluate from step (iv) to said hydrophobic interaction chromatographic material;
(vi) eluting unbound material from said hydrophobic interaction chromatographic material with buffer B, which buffer B is suitable for eluting material not bound to the hydrophobic interaction chromatographic material; and
(vii) eluting said FXI polypeptide from said hydrophobic interaction chromatographic material by gradient-elution with buffer B′, which buffer B′ is suitable for eluting FXI polypeptide from said hydrophobic interaction chromatographic material.

In one embodiment, the present invention concerns a method for purifying a FXI polypeptide from a biological material, the method comprising the steps of: subjecting the eluate from step (vii) or a fluid prepared by use of the eluate from step (vii) to chromatography using a Hydroxyapatite chromatographic material, said chromatography comprising:
(viii) applying the eluate from step (vii) or a fluid prepared by use of the eluate from step (vii) to said hydroxyapatite chromatographic material;
(ix) eluting unbound material from the hydroxyapatite chromatographic material with buffer C, which buffer C is suitable for eluting material not bound to the hydroxyapatite chromatographic material; and
(x) eluting said FXI polypeptide from said hydroxyapatite chromatographic material by gradient-elution with buffer C′, which buffer C′ is suitable for eluting FXI polypeptide from said hydroxyapatite chromatographic material.

In one embodiment, the present invention concerns a method for purifying a FXI polypeptide from a biological material, the method comprising the steps of:
(a) subjecting a biological material comprising a FXI polypeptide to chromatography on a first cation-exchange chromatographic material, said chromatography comprising:
   (i) applying said biological material to said first cation-exchange chromatographic material;
   (ii) eluting unbound material from the first cation-exchange chromatographic material with a buffer A, which buffer A is suitable for eluting material not bound to the first cation-exchange chromatographic material; and
   (iii) eluting unbound material from the first cation-exchange chromatographic material with a buffer A′, which buffer A′ is suitable for eluting material not bound to the first cation-exchange chromatographic material; and
   (iv) eluting said FXI polypeptide from the first cation-exchange chromatographic material by elution with buffer A′, which buffer A″ is suitable for eluting said FXI polypeptide from said first cation-exchange chromatographic material;
(b) subjecting the eluate from step (iv) or a fluid prepared by use of the eluate from step (iv) to chromatography using a hydrophobic interaction chromatographic material, said chromatography comprising:
   (v) applying the eluate from step (iv) or a fluid prepared by use of the eluate from step (iv) to said hydrophobic interaction chromatographic material;
   (vi) eluting unbound material from said hydrophobic interaction chromatographic material with buffer B, which buffer B is suitable for eluting material not bound to the hydrophobic interaction chromatographic material; and
   (vii) eluting said FXI polypeptide from said hydrophobic interaction chromatographic material by gradient-elution with buffer B′, which buffer B′ is suitable for eluting FXI polypeptide from said hydrophobic interaction chromatographic material;
(c) subjecting the eluate from step (vii) or a fluid prepared by use of the eluate from step (vii) to chromatography using a Hydroxyapatite chromatographic material, said chromatography comprising:
   (viii) applying the eluate from step (vii) or a fluid prepared by use of the eluate from step (vii) to said hydroxyapatite chromatographic material;
   (ix) eluting unbound material from the hydroxyapatite chromatographic material with buffer C, which buffer C is suitable for eluting material not bound to the hydroxyapatite chromatographic material; and
   (x) eluting said FXI polypeptide from said hydroxyapatite chromatographic material by gradient-elution with buffer C′, which buffer C′ is suitable for eluting FXI polypeptide from said hydroxyapatite chromatographic material.

Purification of a FXI polypeptide is the process of increasing the concentration of the FXI polypeptide in a sample in relation to other components of said sample, resulting in an increase of the purity of the FXI polypeptide. It should be understood that the concentration of a FXI polypeptide in a sample in relation to other components of said sample is not equivalent to the concentration of FXI polypeptide in the sample. The increase in the purity of the FXI polypeptide may be followed measured by use of methods known in the art, such as for instance by use of SDS-PAGE (Sodium Dodecyl Sulfate Polyacrylamide Gel Electrophoresis), HPLC (High Performance Liquid Chromatography) or Berichrome assays (Dade Behring Diagnostics), or Clot activity assay.

Biological material may be any material derived from or containing cells, cell components or cell products. A biological material may be a biological fluid.

A biological fluid may be any fluid derived from or containing cells, cell components or cell products. Biological fluids include, but are not limited to cell cultures, cell culture supernatants, cell lysates, cleared cell lysates, cell extracts, tissue extracts, blood, plasma, serum, all of which may also be homogenizates and filtrates, and fractions thereof, for instance collected by chromatography of unfractionated biological fluids.

The FXI polypeptides may be purified from a wide variety of biological materials, including cell culture supernatants, which naturally produce a FXI polypeptide, but also of cells which have been genetically modified to produce a FXI polypeptide, such as mammalian cells (for instance CHO cells) transformed with DNA coding for a FXI polypeptide.

The biological material may be treated by use of a number of methods prior to application on the first cation-exchange chromatographic material. Such methods include, but a not limited to, centrifugation, filtration. In one embodiment, the biological material is a biological fluid. In one embodiment of the present invention, the biological fluid is the supernatant of a cell lysate. In one embodiment of the present invention, the biological fluid is the supernatant of a yeast cell lysate.

In one embodiment of the present invention, the FXI polypeptide is purified from a cell culture, such as a mammalian cell culture, as described above. Prior to the chromatography in step (a), the mammalian cells may be separated from cell culture supernatant by centrifugation and/or filtration. Inhibitors such as EDTA (ethylenediamine tetraacetic acid) and benzamidineHCl may be included before being subjected to chromatographic step (a).

A buffer is a solution comprising a substance, which substance is capable of preventing significant changes in the pH of solutions to which small amounts of acids or bases are added and thereby of maintaining largely the original acidity or basicity of the solution. A buffer usually comprises a weak acid or weak base together with a salt thereof.

The pH of the biological fluid may be adjusted to the pH of buffer A prior the chromatography in step (a), for instance by using 1 M HCl or 1 M NaOH or by other means known in the art.

The first cation-exchange chromatographic material may be any cation-exchange chromatographic material known in the art which is capable of binding a FXI polypeptide under one set of conditions and releasing it under a different set of conditions, such as an cation-exchange chromatographic material comprising a sulphopropyl group. Further non-limitimg examples of cation-exchange chromatographic materials include derivatised dextrans, agarose, cellulose, polyacrylamide, and specialty silicas, such as carboxymetyl. Suitable cation-exchange chromatographic material may be identified by subjecting a biological fluid comprising FXI polypeptide to chromatography on the cation-exchange chromatographic material of choice, collecting fractions and determining the purity and content of the fractions, for instance by use of SDS-PAGE (Sodium Dodecyl Sulfate Polyacrylamide Gel Electrophoresis), HPLC (High Performance Liquid Chromatography), clotactivity or Berichrome assays (Dade Behring Diagnostics), monitoring the absorbance of the eluate at 280 nm and by use of other methods known in the art. Examples of suitable cation-exchange chromatographic materials include, but are not limited to Streamline SP XL (Amersham Biosciences cat no 17-5073), Obelix ST CIEX (Amersham Biosciences cat no 11-0010), Streamline Direct CST (Amersham Biosciences 17-5266), S-Support Unosphere, BioRad cat no 156-0113 or Toyopearl SP-550C Toso Haas cat no 14028. In one embodiement Obelix ST CIEX is used.

The first cation-exchange chromatographic material may be pre-equilibrated with buffer A prior to application of the biological material.

Buffer A may comprise protease inhibitors such as EDTA (ethylenediamine tetraacetic acid) and benzamidineHCl, but other commercially available protease inhibitors may also be used.

In one embodiment of the present invention, the pH of buffer A is between 6.5 and 9. In a further embodiment, the pH of buffer A is between 7 and 9. In a further embodiment, the pH of buffer A is about 8.

In one embodiment of the present invention, the conductivity of buffer A is less than about 40 mS/cm.

Buffer A″ is used for the elution of the FXI. Typically, the concentration of one or more of the components of the buffer used for washing in step (ii), in this case buffer A and A′, is increased or decreased during the course of elution or a new component is added to the buffer, and the concentration of this component. This increase or decrease may take place continuously or in discrete steps as it is known in the art. For elution of material bound to an cation-exchange chromatographic material, it is customary to add a salt, for instance NaCl, to buffer A. This specific cation exchanger can also be used as a hydrophobic interaction chromatographic resin, for this kind of resin it is customary to add a propandiol/glycerol to buffer A creating buffer A′. If both NaCl and Propandiol/glycerol is added to buffer A then FXI can be eluted. The determination of which fractions containing FXI polypeptide to pool for further processing, for instance to exclude undesired impurities eluting at the beginning or the end of the FXI polypeptide elution, is within the knowledge of a person skilled in the art. Likewise, the general art of performing an cation-exchange chromatography with regard to for instance pre-equilibration, elution time, washing, reconstitution of the cation-exchange chromatographic material etc is well-known.

After eluting the FXI polypeptide in step (iv), the eluate containing the FXI polypeptides protease inhibitors such as EDTA (ethylenediamine tetraacetic acid) and Benzamidine is added and then taken to step (v). The eluate may also be kept at, for instance, 4° C. for 24 hours or longer, or at, for instance, −80° C.

The hydrophobic interaction chromatographic material for use in step (b) may be any hydrophobic interaction chromatographic material known in the art, which is capable of binding a FXI polypeptide under one set of conditions and releasing it under a different set of conditions, such as a hydrophobic interaction chromatographic material derivatised with phenyl, butyl or octyl groups, or polyacrylic resins. Non-limiting examples of suitable hydrophobic interaction chromatographic material are Amberchrom™ CG 71 (Tosoh Bioscience), Phenyl Sepharose™ High Performance (Amersham, cat no 17-1082), Phenyl Sepharose™ 6 Fast Flow High Substitution (Amersham, cat no 17-0973), Toyopearl® Butyl 650 (Tosoh Bioscience), Toyopearl® Phenyl (Tosoh Bioscience), Source™ 15Phe (Amersham, cat no 17-0147), Butyl Sepharose™ High Performance High Substitution (Amersham, cat no 17-3100), Octyl-Sepharose™ (Amersham, cat no 17-0946) and Phenyl Sepharose™ High Performance High Substitution (Amersham), and the like. In one embodiment of the present invention, the hydrophobic interaction chromatographic material uses butyl as a ligand.

Buffer B and NaCl may be added to the eluate from stage (iv) or a fluid prepared by use of the eluate from stage (iv) prior to the chromatography in step (b) in an amount of about one to two volumes or more, or a concentrated version of buffer B, comprising the same ingredients as buffer B, but in, e.g., twice the concentration, is added to the eluate from stage (iv) or a fluid prepared by use of the eluate from stage (iv) in an amount corresponding to the strength of the concentrated buffer (a twice-concentrated buffer is added in the amount of 1,5 volumes).

Buffer B may have a pH from about 5 to about 9, for instance about 8. In one embodiment of the present invention, buffer B has a conductivity of more than 25 mS/cm, for instance more than 70 mS/cm. This may be achieved, for example, by use of a phosphate buffer or by other means known in the art, e.g. NaCl. In one embodiment of the present invention, the conductivity of the eluate from step (iv) or a fluid prepared by use of the eluate from step (iv) is adjusted to a conductivity of at least about 60 mS/cm.

Buffer B′ is used for the elution of the FXI polypeptide by gradient elution. In gradient elution, the composition of buffer B′ is changed during the course of elution. Typically, the concentration of one or more of the components of the buffer used for washing in step (vi), in this case buffer B, is increased or decreased during the course of elution, or a new component is added to the buffer and the concentration of this component is then increased during the course of elution. This increase or decrease may take place continuously or in discrete steps, as is well known in the art. For elution of material bound to a hydrophobic interaction chromatographic material, it is customary to dilute the washing buffer with water until at least a major portion of the bound FXI polypeptide is eluted. The determination of which fractions containing FXI polypeptide to pool for further processing, e.g. in order to exclude undesired impurities eluting at the beginning or the end of the FXI polypeptide elution, is within the knowledge of a person skilled in the art. Likewise, the general art of performing a hydrophobic interaction chromatography with regard to, e.g., pre-equilibration, elution time, washing, reconstitution of the hydrophobic interaction chromatographic material, etc., is well known.

In one embodiment of the present invention, the eluate from stage (vii) or a fluid prepared by use of the eluate from stage (vii) is treated by use of a method comprising a step of adding one or more stabilizing agents which are capable of increasing the stability of the FXI polypeptide in an amount effective to significantly improve the stability thereof.

This step, and optionally other steps of post-processing known in the art, may be carried out alone or in combination, and the order in which the steps are performed is not critical. The person skilled in the art will be able to determine how and when to perform these steps.

In one embodiment of the present invention, a stabilizing agents which are capable of increasing the physical and/or chemical stability of the FXI polypeptide is added to the fractions containing FXI.

The term “physical stability” of the FXI polypeptide as used herein refers to the potential tendency of the protein to form biologically inactive and/or insoluble aggregates or multimers of the protein as a result of exposure of the protein to thermo-mechanical stresses and/or interaction with interfaces and surfaces that are destabilizing, such as hydrophobic surfaces and interfaces. Physical stability of the FXI polypeptide when present in buffer A may be evaluated by means of visual inspection and/or turbidity measurements after exposing the formulation filled in suitable containers (e.g. cartridges or vials) to mechanical/physical stress (e.g. agitation) at different temperatures for various time periods.

Visual inspection of the FXI polypeptide when present in buffer may be performed in a sharp focused light with a dark background. The turbidity of the composition may be characterized by a visual score ranking the degree of turbidity, for instance on a scale from 0 to 3 (a composition showing no turbidity then corresponding to a visual score 0, and a composition showing visual turbidity in daylight corresponding to visual score 3). A composition is classified as physically unstable with respect to protein aggregation when it shows visual turbidity in daylight. Alternatively, the turbidity of the composition may be evaluated by simple turbidity measurements well-known to the skilled person, for instance by measuring the optical density of the solution at a wavelength of 405 nm (OD₄₀₅). Physical stability of the aqueous protein compositions may also be evaluated by using a spectroscopic agent or probe of the conformational status of the protein. The probe is preferably a small molecule that preferentially binds to a non-native conformer of the protein. One example of a small-molecule spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as antrhacene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the FXI polypeptide when used herein refers to chemical covalent changes in the protein structure leading to formation of chemical degradation products with potentially lower biological potency and/or potentially increased immunogenic properties compared to the native protein structure. Various chemical degradation products can be formed depending on the type and nature of the native protein and the environment to which the protein is exposed. Elimination of chemical degradation can most probably not be completely avoided, and an increase in amounts of chemical degradation products is often seen during storage and use of the protein composition, as well-known to a person skilled in the art. Most proteins are prone to deamidation, a process in which the side-chain amide group in glutaminyl or asparaginyl residues is hydrolysed to form a free carboxylic acid. Other degradation pathways involve formation of high-molecular-weight transformation products wherein two or more protein molecules are covalently bound to each other via transamidation and/or disulfide interactions, leading to formation of covalently bound dimer, oligomer and polymer degradation products (Stability of Protein Pharmaceuticals, Ahern. T. J. & Manning M. C., Plenum Press, New York 1992). Oxidation (e.g. of methionine residues) can be mentioned as another variant of chemical degradation. The chemical stability of the FXI polypeptide when present in buffer B′ can be evaluated by measuring the amounts of chemical degradation products at various times after exposure to different environmental conditions; the formation of degradation products can, for example, often be accelerated by increase in temperature. The amount of each individual degradation product is often determined by separation of the degradation products depending on molecule size and/or charge using various chromatographic techniques (e.g. SEC-HPLC and/or RP-HPLC).

Any agent which is capable of significantly improving the physical and/or chemical stability of FXI polypeptide when present in buffer B′ (e.g. as determined by measuring turbidity at OD₄₀₅ over a period of time) may be used as a stabilizing agent.

An agent suitable for use as stabilizing agent, for instance, be a salt (e.g. sodium chloride), a sugar, an alcohol (such as an C₄-C₈ alcohol), an alditol, an amino acid (e.g. glycine, histidine, arginine, lysine, isoleucine, aspartic acid, tryptophan or threonine), a polyethyleneglycol (e.g. PEG400), or a mixture of one or more thereof. Any sugar, such as a mono-, di-, or polysaccharide, or a water-soluble glucan, may be used. An alditol is a polyalcohol of structure HOCH₂—[CH(OH)]ₙ—CH₂OH, where n is 1, 2, 3 . . . etc. Non-limiting examples of substances which are sugars, alcohols or alditols are fructose, glucose, mannose, sorbose, xylose, maltose, lactose, sucrose, trehalose, dextran, pullulan, dextrin, cyclodextrin, soluble starch, hydroxyethyl starch, carboxymethylcellulose-Na, mannitol, sorbitol, inositol, galactitol, dulcitol, xylitol, arabitol, glycerol (glycerine), propan-1,2-diol (propylene glycol), propan-1,3-diol, and butan-1,3-diol. The sugars, alcohols and alditols mentioned above may be used individually or in combination. There is no fixed limit to the amount used, as long as the substance is soluble in the liquid preparation and improves the physical stability of a FXI polypeptide in solution. In this respect, reference is made to Remington: The Science and Practice of Pharmacy, 19th edition, 1995.

In one embodiment of the present invention, one or more stabilizing agents of the polyalcohol type is added.

In one embodiment of the present invention, one or more stabilizing agents selected from the group consisting of glycerol (propan-1,2,3-triol), propylene glycol (propan-1,2-diol), propan-1,3-diol, propyl alcohol (1-propanol) and isopropyl alcohol (2-propanol) is added. In one embodiment of the present invention, one or more stabilizing agents selected from the group consisting of glycerol, propylene glycol and propan-1,3-diol is added.

In a further embodiment of the present invention, when the stabilizing is a liquid alcohol or liquid polyalcohol [such as, e.g., glycerol, propylene glycol, propan-1,3-diol, propyl alcohol or isopropyl alcohol], the stabilizing agent is present in a concentration of from about 5% by volume (v/v) to about 50% (v/v). In a further embodiment, a stabilizing agent of the liquid alcohol or liquid polyalcohol type is present in a concentration of from about 10% (v/v) to about 50% (v/v). In a further embodiment, a stabilizing agent of the liquid alcohol or liquid polyalcohol type is present in a concentration of from about 10% (v/v) to about 20% (v/v). In a further embodiment, a stabilizing agent of the liquid alcohol or liquid polyalcohol type is present in a concentration of about 10% (v/v). In a still further embodiment, a stabilizing agent of the liquid alcohol or liquid polyalcohol type is present in a concentration of about 20% (v/v).

The stabilizing agent mentioned should be capable of increasing the physical and/or chemical stability, as described above, of the FXI polypeptide. Any agent which is capable of significantly improving the physical and/or chemical stability of FXI polypeptide (e.g. as determined by measuring turbidity at OD₄₀₅ over a period of time) may be used as a stabilizing agent.

The eluate from step (vii) may be used for the preparation of a pharmaceutical composition. This may involve a change of buffer, and/or adjustment of the conductivity and/or pH to physiological values, and/or other actions to render the eluate acceptable for use in mammals, such as humans; means of rendering such an eluate acceptable for use in this manner are well known in the art. The eluate may also be kept at, e.g., 4° C. for 24 hours or longer, or at, e.g., −80° C.

In one embodiment of the present invention, the method further comprises a step of subjecting the eluate from stage (vii), or a fluid prepared by use of the eluate from stage (vii), to chromatography on a hydroxyapatite chromatographic material, said chromatography comprising:
(viii) applying the eluate from stage (vii), or a fluid prepared by use of the eluate from stage (vii), to said hydroxyapatite chromatographic material;
(ix) eluting unbound material from the hydroxyapatite chromatographic material with buffer C, which buffer C is suitable for eluting material not bound to the hydroxyapatite chromatographic material; and
(x) eluting said FXI polypeptide from the hydroxyapatite chromatographic material with buffer C′, which buffer C′ is suitable for eluting FXI polypeptides which bind to the hydroxyapatite chromatographic material in step (ix).

A fluid prepared by use of the eluate from stage (vii) may, for instance, be prepared before application.

In one embodiment of the present invention, the conductivity of the eluate from stage (vii), or a fluid prepared by use of the eluate from stage (vi), is adjusted to less than about 20 mS/cm by adding water. pH is adjusted to 5,8 to 9. In one embodiment pH is adjusted to 6,0

The components of buffer C and buffer C′ may be chosen with a view to the desired final pharmaceutical composition of the FXI polypeptide. Such considerations are within the knowledge of a person skilled in the art.

In one embodiment of the present invention, buffer C comprises one or more stabilizing agents, which stabilizing agents are capable of increasing the physical and/or chemical stability, as described above, of the FXI polypeptide. Any agent which is capable of significantly improving the physical and/or chemical stability of FXI polypeptide when present in buffer C (e.g. as determined by measuring turbidity at OD₄₀₅ over a period of time) may be used as a stabilizing agent in buffer C or buffer C′.

An agent suitable for use as stabilizing agent in buffer C may, for instance, be a salt (e.g. sodium chloride), a sugar, an alcohol (such as an C₄-C₈ alcohol), an alditol, an amino acid (e.g. glycine, histidine, arginine, lysine, isoleucine, aspartic acid, tryptophan or threonine), a polyethyleneglycol (e.g. PEG400), or a mixture of one or more thereof. Any sugar, such as a mono-, di-, or polysaccharide, or a water-soluble glucan, may be used. Non-limiting examples of substances which are sugars, alcohols or alditols are fructose, glucose, mannose, sorbose, xylose, maltose, lactose, sucrose, trehalose, dextran, pullulan, dextrin, cyclodextrin, soluble starch, hydroxyethyl starch, carboxymethylcellulose-Na, mannitol, sorbitol, inositol, galactitol, dulcitol, xylitol, arabitol, glycerol (glycerine), propan-1,2-diol (propylene glycol), propan-1,3-diol, and butan-1,3-diol. The sugars, alcohols and alditols mentioned above may be used individually or in combination. There is no fixed limit to the amount used, as long as the substance is soluble in the liquid preparation and improves the physical stability of a FXI polypeptide in solution. In this respect, reference is made to Remington: The Science and Practice of Pharmacy, 19th edition, 1995.

In one embodiment of the present invention, buffer C comprises one or more stabilizing agents of the polyalcohol type.

In one embodiment of the present invention, buffer C comprises NaCl.

One or more stabilizing agents selected from the group consisting of glycerol (propan-1,2,3-triol), propylene glycol (propan-1,2-diol), propan-1,3-diol, propyl alcohol (1-propanol) and isopropyl alcohol (2-propanol) is added to the fluid from (x).

In one embodiment of the present invention, one or more stabilizing agents selected from the group consisting of glycerol, propylene glycol and propan-1,3-diol is added. In one embodiment of the present invention, propylene glycol is added.

In a further embodiment of the present invention, when the stabilizing agent it is present in a concentration of from about 5% (v/v) to about 50% (v/v). In a further embodiment, a stabilizing agent of the liquid alcohol or liquid polyalcohol type is present in a concentration of from about 10% (v/v) to about 50% (v/v). In a further embodiment, a stabilizing agent of the liquid alcohol or liquid polyalcohol type used is present in a concentration of from about 10% (v/v) to about 20% (v/v). In a further embodiment, a stabilizing agent of the liquid alcohol or liquid polyalcohol type is present in a concentration of about 10% (v/v).

Buffer C′ is used for the elution of the FXI polypeptide by gradient elution, wherein the composition of buffer C′ is changed during the course of elution. Typically, the concentration of one or more of the components of the buffer used for washing in step (ix), in this case buffer C, is increased or decreased during the course of elution, or a new component is added to the buffer and the concentration of this component is then increased during the course of elution. This increase or decrease may take place continuously or in discrete steps, as is well known in the art. For elution of material bound to hydroxyapatite chromatographic material, it is customary to add a salt, e.g. K—PO₄ or NaCl, to buffer C and then increase the concentration of the salt until at least a major portion of the bound FXI polypeptide is eluted. The determination of which fractions containing FXI polypeptide to pool for further processing, e.g. in order to exclude undesired impurities eluting at the beginning or the end of the FXI polypeptide elution, is within the knowledge of a person skilled in the art. Likewise, the general art of performing an hydroxyapatite chromatography with regard to, e.g., pre-equilibration, elution time, washing, reconstitution of the cation-exchange chromatographic material, etc., is well known.

In a series of embodiments, the use of one or more stabilizing agents in any or all of the solutions used in purification of FXI results in an increase in the physical and/or chemical stability of FXI by at least 10%, 25%, 50%, or 100% over the physical and/or chemical stability of a control (i.e., FXI subjected to the same treatment but in the absence of the stabilizing agent). In another series of embodiments, the use of one or more stabilizing agents in any or all of the solutions used in purification of FXI results in an increase in the physical and/or chemical stability of FXI by at least 2-fold, 5-fold, 10-fold, or 20-fold over the physical and/or chemical stability of a control (i.e., FXI subjected to the same treatment but in the absence of the stabilizing agent).

Activation of FXI

Wild-type human FXI is normally activated by proteolytic cleavage between Arg₃₆₀ and IIe₃₇₀, which may be catalyzed, e.g., by FXIa, FXIIa, or thrombin. If desired, activation of9 FXI for use in the present invention may be achieved using FXIa or FXIIa (both from Enzyme Research Laboratories, South Bend, Ind.) orthrombin (Sigma). See, e.g., Sun et al. (1999) J. Biol Chem 51:36373-36373 and Baglia (2003) J. Biol Chem 24:21744-21750. It is also within the scope of the invention to utilize other proteases to activate FXI polypeptides and in particular, FXI-related polypeptides.

The present invention encompasses methods and compositions for the therapeutic administration of FXI that utilize preparations having different FXI activation levels. In some embodiments, the methods and compositions employ FXI polypeptides that have not been subjected to any activation procedure. In some embodiments, the preparation of FXI or FXI-related polypeptide exhibits a ratio (by mass) of activated:zymogen FXI or FXI-related polypeptide of between about 1:99 to about 99:1, such as, e.g., between about 5:95 to about 95:5; about 10:90 to about 90:10; about 20:80 to about 80:20; about 30:70 to about 70:30; about 40:60 to about 60:40; and about 50:50. In some embodiments, the preparation contains not more than about 5% FXIa relative to the total FXI on a molar basis; more preferably, not more than about 2.5%, even more preferably, not more than about 1%, most preferably not more than about 0.5% or 0.1%. In some embodiments, the preparation contains not more than about 0.01-0.05% FXIa on a molar basis. In some embodiments, the preparation contains not more than about 0.01-0.04% FXIa on a molar basis. In some embodiments, the preparation contains not more than about 0.01-0.03% FXIa on a molar basis.

The invention also relates to FXI-related polypeptides that exhibit a differential capacity to be activated relative to wild-type FXI, such as, e.g., FXI-related polypeptides that are more easily activated by FXIIa than by thrombin, and vice versa; polypeptides that are constitutively activated, even in the absence of proteolytic cleavage; heterodimers in which one monomer (by virtue of mutation or chemical modification) cannot be proteolytically activated; and the like.

Furthermore, the invention also relates to FXI-related polypeptides that are resistant to autoactivation, i.e. variants where the ratio between rate of activation by thrombin (and/or FXI Ia) versus rate of activation by FXIa is higher than for wild-type FXI.

The methods and compositions of the invention may also employ treatment, pre-treatment, storage, or co-administration of a FXI polypeptide with additional agents that inhibit and/or promote activation. Non-limiting examples of agents that inhibit activation include C1 esterase inhibitor (C1Inb), α-2 antiplasmin, (α2AP), α1-antitrypsin (α1 AT), protease Nexin II, benzamidine, heparin, and antithrombin III; non-limiting examples of agents that promote activation include FXIa, FXIIa, and thrombin.

Pharmaceutical Formulations Comprising FXI:

The present invention encompasses pharmaceutical compositions comprising a preparation of FXI or FXI-related polypeptide for prophylactic and/or therapeutic treatment.

Pharmaceutical compositions or formulations according to the invention comprise a a FXI polypeptide, such as, e.g., at concentrations between 0.001-100 mg/ml, that is preferably dissolved in a pharmaceutically acceptable carrier, preferably an aqueous carrier or diluent. Briefly, pharmaceutical compositions suitable for use according to the present invention are made by mixing a preparation comprising FXI and/or a FXI-related polypeptide, preferably in purified form, with suitable adjuvants and a suitable carrier or diluent. A variety of aqueous carriers may be used, such as water, buffered water, 0.4% saline, 0.3% glycine, sugars, detergents, salts, buffers, glycerols, preservatives, protease inhibitors, glycols, and the like. The preparations of the invention can also be formulated using non-aqueous carriers, such as, e.g., in the form of a gel or as liposome preparations for delivery or targeting to the sites of injury. Liposome preparations are generally described in, e.g., U.S. Pat. Nos. 4,837,028, 4,501,728, and 4,975,282. The compositions may be sterilised by conventional, well-known sterilisation techniques. The resulting aqueous solutions may be packaged for use or filtered under aseptic conditions and lyophilised, the lyophilised preparation being combined with a sterile aqueous solution prior to administration.

The compositions may contain pharmaceutically acceptable auxiliary substances or adjuvants, including, without limitation, pH adjusting and buffering agents, tonicity adjusting agents, preservatives, stabilizers, surfactants, chelating agents, and the like. One skilled in this art may formulate the compositions of the invention an appropriate manner, and in accordance with accepted practices, such as those disclosed in Remington's Pharmaceutical Sciences, Gennaro, ed., Mack Publishing Co., Easton, Pa., 1990.

In one embodiment of the invention, the pharmaceutical compositions comprising a preparation of FXI or FXI-related polypeptide further comprises a pH adjusting and buffering agent. In one embodiment of the invention, the pharmaceutical compositions comprising a preparation of FXI or FXI-related polypeptide further comprises a tonicity adjusting agent. In one embodiment of the invention, the pharmaceutical compositions comprising a preparation of FXI or FXI-related polypeptide further comprises a preservative. In one embodiment of the invention, the pharmaceutical compositions comprising a preparation of FXI or FXI-related polypeptide further comprises a stabilizer. In one embodiment of the invention, the pharmaceutical compositions comprising a preparation of FXI or FXI-related polypeptide further comprises a surfactant. In one embodiment of the invention, the pharmaceutical compositions comprising a preparation of FXI or FXI-related polypeptide further comprises a chelating agent.

Non-limiting examples of suitable buffers include acetate buffers, carbonate buffers, citrate buffers, glycylglycine buffers, histidine buffers, glycine buffers, lysine buffers, arginine buffers, phosphate buffers (containing, e.g.,sodium dihydrogen phosphate, disodium hydrogen phosphate or trisodium phosphate), TRIS [tris(hydroxymethyl)aminomethane] buffers, bicine buffers, tricine buffers, malate buffers, succinate buffers, maleate buffers, fumarate buffers, tartrate buffers, aspartate buffers, and mixtures thereof.

Non-limiting examples of pharmaceutically acceptible preservatives include phenol, o-cresol, m-cresol, p-cresol, chlorocresol, methyl p-hydroxybenzoate, ethyl p-hydroxybenzoate, propyl p-hydroxybenzoate, butyl p-hydroxybenzoate, 2-phenoxyethanol, 2-phenylethanol, benzyl alcohol, chlorobutanol, thiomerosal, bronopol, benzoic acid, imidurea, chlorohexidine, sodium dehydroacetate, benzethonium chloride, chlorphenesine (3-p-chlorphenoxypropane-1,2-diol), benzamidine and mixtures thereof. In a further embodiment of the present invention the preservative is present in a concentration from 0.1 mg/ml to 20 mg/ml. In one further embodiment of the present invention the preservative is present in a concentration from 0.1 mg/ml to 5 mg/ml. In another further embodiment of the present invention the preservative is present in a concentration from 5 mg/ml to 10 mg/ml. In another further embodiment of the present invention the preservative is present in a concentration from 10 mg/ml to 20 mg/ml. The use of a preservative in pharmaceutical compositions is well-known to the skilled person (see, e.g., Remington: The Science and Practice of Pharmacy, 19th edition, 1995).

Non-limiting examples of tonicity-adjusting agents (which are normally incorporated for the purpose of rendering the formulation substantially isotonic include salts (e.g. sodium chloride), sugars, alcohols (such as C₄-C₈ alcohols), alditols, amino acids (e.g. glycine, histidine, arginine, lysine, isoleucine, aspartic acid, tryptophan or threonine), polyethyleneglycols (e.g. PEG400), and mixtures thereof. Any sugar, such as a mono-, di-, or polysaccharide, or a water-soluble glucan, may be used. Non-limiting examples of substances which are sugars, alcohols or alditols are fructose, glucose, mannose, sorbose, xylose, maltose, lactose, sucrose, trehalose, dextran, pullulan, dextrin, cyclodextrin, soluble starch, hydroxyethyl starch, carboxymethylcellulose-Na, mannitol, sorbitol, inositol, galactitol, dulcitol, xylitol, arabitol, glycerol (glycerine), propan-1,2-diol (propylene glycol), propan-1,3-diol, and butan-1,3-diol. The sugars, alcohols and alditols mentioned above may be used individually or in combination. There is no fixed limit to the amount used, as long as the substance is soluble in the liquid preparation.

In one embodiment, the tonicity-adjusting agent is present in a concentration of from about 1 mg/ml to about 150 mg/ml. In a further embodiment of the present invention, the tonicity-adjusting agent is present in a concentration of from about 1 mg/ml to about 50 mg/ml. In one embodiment, the tonicity-adjusting agent is NaCl. In one embodiment, the tonicity-adjusting agent is NaCl present in a concentration of from about 1 mg/ml to about 150 mg/ml. In a further embodiment of the present invention, the tonicity-adjusting agent is NaCl present in a concentration of from about 1 mg/ml to about 50 mg/ml.

Non-limiting examples of chelating agents include salts of EDTA, citric acid and aspartic acid, and mixtures thereof. In some embodiments, a chelating agent is present in a concentration from 0.1 mg/ml to 5 mg/ml; from 0.1 mg/ml to 2 mg/ml; or from 2 mg/ml to 5 mg/ml.

The pharmaceutical compositions of the present invention may include as a therapeutically active component a polypeptide that possibly may exhibit aggregate formation during storage in liquid pharmaceutical compositions. The term “aggregate formation” is intended to indicate a physical interaction between the polypeptide molecules that results in formation of oligomers which may remain soluble, or of large visible aggregates that precipitate from the solution. The term “during storage” refers to a liquid pharmaceutical composition or formulation which, once prepared, is not immediately administered to a subject. Rather, following preparation, it is packaged for storage in a liquid form, in a frozen state, or in a dried form for later reconstitution into a liquid form or other form suitable for administration to a subject. The term “dried form” refers to a liquid pharmaceutical composition or formulation dried by freeze-drying [i.e. lyophilization; see, for example, Williams and Polli (1984), J. Parenteral Sci. Technol. 38:48-59], by spray-drying [see Masters (1991) in Spray-Drying Handbook (5th ed; Longman Scientific and Technical, Essex, U.K.), pp. 491-676; Broadhead et al. (1992) Drug Devel. Ind. Pharm. 18:1169-1206; and Mumenthaler et al. (1994) Pharm. Res. 11:12-20] or by air-drying [Carpenter and Crowe (1988), Cryobiology 25:459-470; and Roser (1991) Biopharm. 4:47-53]. Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems, such as blockage of tubing, membranes or pumps when the polypeptide-containing pharmaceutical composition is administered using an infusion system.

In one embodiment of the present invention, the pharmaceutical composition comprises an amount of an amino acid base sufficient to decrease aggregate formation by the polypeptide during storage of the composition. The term “amino acid base” indicates an amino acid or a combination of amino acids where any given amino acid is present either in its free base form or in its salt form. When a combination of amino acids is used, all of the amino acids may be present in their free base forms, all may be present in their salt forms, or some may be present in their free base forms while others are present in their salt forms. In one embodiment, amino acids for use in preparing compositions of the present invention are those carrying a charged side chain, such as arginine, lysine, aspartic acid or glutamic acid. Any stereoisomer of a particular amino acid (e.g. glycine, methionine, histidine, arginine, lysine, isoleucine, aspartic acid, tryptophan, threonine or a mixture of one or more thereof), or combinations of these stereoisomers, may be present in pharmaceutical compositions of the present invention so long as the particular amino acid is present either in its free base form or its salt form. In one embodiment the L-stereoisomer is used. Compositions of the present invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the present invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. The compound imidazole is also to be regarded as an amino acid analogue in the context of the present invention. Typcally, the amino acids or amino acid analogues are used in a concentration which is sufficient to prevent or delay aggregation of the protein.

In one embodiment, the pharmaceutical formulation comprises methionine (or another sulfur-containing amino acid or amino acid analogue) to inhibit oxidation of methionine residues to their sulfoxide form when the factor XI polypeptide is a polypeptide comprising at least one methionine residue susceptible to such oxidation. The term “inhibit oxidation” is intended to indicate minimization of accumulation of oxidized species (of methionine) with time. Inhibition of methionine oxidation results in greater retention of the polypeptide in its proper molecular form. Any stereoisomer of methionine (L, D or DL isomer) or combinations thereof can be used. The amount to be added should be an amount sufficient to inhibit oxidation of the methionine residues such that the amount of sulfoxide form of methionine is acceptable to regulatory agencies. Typically, this means that the composition contains no more than from about 10% to about 30% methionine sulfoxide form. This can in general be achieved by adding methionine in an amount such that the ratio of added methionine to methionine residues ranges from about 1:1 to about 1000:1, such as 10:1 to about 100:1.

Non-limiting examples of stabilizers include high-molecular-weight polymers or low-molecular-weight compounds, such as, e.g., polyethylene-glycols (e.g. PEG 3350), polyvinyl alcohol (PVA), polyvinylpyrrolidone, carboxy-/hydroxycellulose and derivatives thereof (including HPC, HPC-SL, HPC-L and HPMC), cyclodextrins, sulfur-containing substances as monothioglycerol, thioglycolic acid and 2-methylthioethanol, various salts (e.g. sodium chloride), glycerol, propylene glycol, propan-1,3-diol, propyl alcohol (1-propanol) and isopropyl alcohol (2-propanol).

Non-limiting examples of surfactants include detergents, ethoxylated castor oil, polyglycolyzed glycerides, acetylated monoglycerides, sorbitan fatty acid esters, polyoxypropylene-polyoxyethylene block polymers (e.g. poloxamers such as Pluronic® F68, poloxamer 188 and 407, Triton X-100 ), polyoxyethylene sorbitan fatty acid esters, polyoxyethylene and polyethylene derivatives such as alkylated and alkoxylated derivatives (“Tweens”, e.g. Tween-20, Tween-40, Tween-80 and Brij-35), monoglycerides and ethoxylated derivatives thereof, diglycerides and polyoxyethylene derivatives thereof, alcohols, glycerol, lectins and phospholipids (eg. phosphatidyl-serine, phosphatidyl-choline, phosphatidyl-ethanolamine, phosphatidyl-inositol, diphosphatidyl-glycerol and sphingomyelin), derivatives of phospholipids (e.g. dipalmitoyl-phosphatidic acid) and lysophospholipids (e.g. palmitoyl lysophosphatidyl-L-serine and 1-acyl-sn-glycero-3-phosphate esters of ethanolamine, choline, serine or threonine), and alkyl-, alkoxyl- (alkyl ester) and alkoxy- (alkyl ether) derivatives of lysophosphatidyl and phosphatidylcholines, e.g. lauroyl and myristoyl derivatives of lysophosphatidylcholine, dipalmitoylphosphatidylcholine, and modifications of the polar head group, i.e. cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (e.g. cephalins), glyceroglycolipids (e.g. galactopyranoside), sphingoglycolipids (e.g. ceramides, gangliosides), dodecylphosphocholine, hen egg lysolecithin, fusidic acid derivatives (e.g. sodium tauro-dihydrofusidate etc.), long-chain fatty acids [e.g. C₆-Cl₂ fatty acids (such as oleic acid or caprylic acid)] and salts thereof, acylcarnitines and derivatives thereof, N-acylated derivatives of lysine, arginine and histidine, side-chain acylated derivatives of lysine and arginine, N^{α}-acylated derivatives of dipeptides comprising any combination of lysine, arginine and histidine and a neutral or acidic amino acid, N-acylated derivatives of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulfate or sodium lauryl sulfate), sodium caprylate, cholic acid and derivatives thereof, bile acids and salts thereof, and glycine or taurine conjugates, ursodeoxycholic acid, sodium cholate, sodium deoxycholate, sodium taurocholate, sodium glycocholate, N-hexadecyl-N,N-dimethyl-3-ammonio-1-propanesulfonate, anionic (alkyl-aryl-sulfonates) monovalent surfactants, zwitterionic surfactants (e.g. N-alkyl-N,N-dimethylammonio-1-propanesulfonates, 3-cholamido-1-propyidimethylammonio-1-propanesulfonate), cationic surfactants (quaternary ammonium bases; e.g. cetyl-trimethylammonium bromide, cetylpyridinium chloride), non-ionic surfactants (eg. Dodecyl ,-D-glucopyranoside), and poloxamines (eg. Tetronic's), i.e. tetrafunctional block copolymers derived from sequential addition of propylene oxide and ethylene oxide to ethylenediamine; or the surfactant may be selected from the group of imidazoline derivatives, or mixtures thereof. In one embodiment, the pharmaceutical formulation comprises a surfactant in a concentration of about 0.01 mg/ml to about 50 mg/ml. In one embodiment, the pharmaceutical formulation comprises Tween-80. In one embodiment, the pharmaceutical formulation comprises poloxamer 188.

In one embodiment, the pharmaceutical formulation comprises an electrolyte. In one embodiment, the pharmaceutical formulation comprises an electrolyte, such as NaCl. In one embodiment, the pharmaceutical formulation comprises an electrolyte, such as KCl.

In one embodiment an electrolyte, such as NaCl, such as in a concentration of 150 mM, is employed when Tween 80 is employed as stabiliser. In one embodiment aggregation following storage is avoided.

In a series of embodiments, the use of one or more stabilizing agents used in a pharmaceutical formulation comprising a preparation of FXI or FXI-related polypeptide results in an increase in the physical and/or chemical stability of FXI by at least 10%, 25%, 50%, or 100% over the physical and/or chemical stability of a control (i.e., FXI subjected to the same treatment but in the absence of the stabilizing agent). In another series of embodiments, the use of one or more stabilizing agents in a pharmaceutical formulation comprising a preparation of FXI or FXI-related polypeptide results in an increase in the physical and/or chemical stability of FXI by at least 2-fold, 5-fold, 10-fold, or 20-fold over the physical and/or chemical stability of a control (i.e., FXI subjected to the same treatment but in the absence of the stabilizing agent).

The following table provides non-limiting examples of suitable formulations. No aggregation was observed after at least a month at ambient temperature when such formulations contained 1.7 mg/ml FXI.

| | | | Isotonic | | Antimicrobial |
|---|---|---|---|---|---|
| ID | pH | Buffer | agent | Stabiliser | preservative |
| A1 | 8.5 | 50 mM | na | na | na |
| | | TRIS(HYDROXYMETHYL) | | | |
| | | AMINOMETHANE | | | |
| | | (TRIS), pH 8.5 | | | |
| A2 | 8.5 | 50 mM | 150 mM NaCl | na | na |
| | | TRIS(HYDROXYMETHYL) | | | |
| | | AMINOMETHANE | | | |
| | | (TRIS), pH 8.5 | | | |
| A3 | 8.5 | 50 mM | na | 0.001% w/v | na |
| | | TRIS(HYDROXYMETHYL) | | Tween 80 | |
| | | AMINOMETHANE | | | |
| | | (TRIS), pH 8.5 | | | |
| A4 | 8.5 | 50 mM | na | 0.1% w/v | na |
| | | TRIS(HYDROXYMETHYL) | | Tween 80 | |
| | | AMINOMETHANE | | | |
| | | (TRIS), pH 8.5 | | | |
| A5 | 8.5 | 50 mM | 150mM NaCl | 0.1% | na |
| | | TRIS(HYDROXYMETHYL) | | Tween 80 | |
| | | AMINOMETHANE | | | |
| | | (TRIS), pH 8.5 | | | |
| A6 | 8.5 | 50 mM | 16.0 mg/ml | na | 0.5 w/v % phenol |
| | | TRIS(HYOROXYMETHYL) | glycerol | | |
| | | AMINOMETHANE | | | |
| | | (TRIS), pH 8.5 | | | |
| A7 | 8.5 | 50 mM | na | 5 w/v % | na |
| | | TRIS(HYDRQXYMETHYL) | | hydroxypropyl- | |
| | | AMINOMETHANE | | betacyclo- | |
| | | (TRIS), pH 8.5 | | dextrin | |
| A8 | 8.5 | 50 mM | na | 0.1 w/v % | na |
| | | TRIS(HYDROXYMETHYL) | | human serum | na |
| | | AMINOMETHANE | | albumin | |
| | | (TRIS), pH 8.5 | | | |
| A9 | 8.5 | 50 mM | na | 0.5M | na |
| | | TRIS(HYDROXYMETHYL) | | Sucrose | |
| | | AMINOMETHANE | | | |
| | | (TRIS), pH 8.5 | | | |
| A10 | 8.5 | 50 mM | na | 0.3 w/v % | na |
| | | TRIS(HYDROXYMETHYL) | | Poloxamer 188 | |
| | | AMINOMETHANE | | | |
| | | (TRIS). pH 8.S | | | |
| A11 | 8.5 | 50 mM | na | 18.6 mg/ml | |
| B1 | 8.0 | TRIS(HYDROXYMETHYL) | | EDTA | |
| | | AMINOMETHANE | 18.6 mg/mI | | |
| | | (TRIS), pH 8.S | | | |
| | | 50 mM | na | na | na |
| | | TRIS(HYDROXYMETHYL) | | | |
| | | AMINOMETHANE | | | |
| | | (TRIS), pH 8.0 | | | |
| B2 | 8.0 | 50 mM | 150 mM NaCl | na | na |
| | | TRIS(HYDROXYMETHYL) | | | |
| | | AMINOMETHANE | | | |
| | | (TRIS), pH 8.0 | | | |
| B3 | 8.0 | 50 mM | na | 0.001% w/v | na |
| | | TRIS(HYDROXYMETHYL) | | Tween 80 | |
| | | AMINOMETHANE | | | |
| | | (TRIS), pH 8.0 | | | |
| B4 | 8.0 | 50 mM | na | 0.1% w/v | na |
| | | TRIS(HYDROXYMETHYL) | | Tween 80 | |
| | | AMINOMETHANE | | | |
| | | (TRIS), pH 8.0 | | | |
| B5 | 8.0 | 50 mM | 150 mM NaCl | 0.1% | na |
| | | TRIS(HYDROXYMETHYL) | | Tween 80 | |
| | | AMINOMETHANE | | | |
| | | (TRIS), pH 8.0 | | | |
| B6 | 8.0 | 50 mM | 16.0 mg/ml | na | 0.5 w/v % phenol |
| | | TRIS(HYDROXYMETHYL) | glycerol | | |
| | | AMINOMETHANE | | | |
| | | (TRIS), pH 8.0 | | | |
| B7 | 8.0 | 50 mM | na | 5 w/v % | na |
| | | TRIS(HYDROXYMETHYL) | | hydroxypropyl- | |
| | | AMINOMETHANE | | betacylo- | |
| | | (TRIS), pH 8.0 | | dextrin | |
| B8 | 8.0 | 50 mM | na | 0.1 w/v % | na |
| | | TRIS(HYDROXYMETHYL) | | human serum | |
| | | AMINOMETHANE | | albumin | |
| | | (TRIS), pH 8.0 | | | |
| B9 | 8.0 | 50 mM | na | 0.5 M | na |
| | | TRIS(HYDROXYMETHYL) | | Sucrose | |
| | | AMINOMETHANE | | | |
| | | (TRIS), pH 8.0 | | | |
| B10 | 8.0 | 50 mM | na | 0.3 w/v % | na |
| | | TRIS(HYDROXYMETHYL) | | Poloxamer 188 | |
| | | AMINOMETHANE | | | |
| | | (TRIS), pH 8.0 | | | |
| B11 | 8.0 | 50 mM | na | 18.6 mg/ml | |
| | | TRIS(HYDROXYMETHYL) | | EDTA | |
| | | AMINOMETHANE | | | |
| | | (TRIS), pH 8.0 | | | |
| B12 | 6.1 | Histidine 1.36 mg/ml, pH 6.1 | 40 mg/ml mannitol | 0.3 w/v % | 6 mg/ml phenol |
| | | | | Poloxamer 188 | |
| C1 | 7.4 | 50 mM phosphate, pH 7.4 | na | na | na |
| C2 | 7.4 | 50 mM phosphate, pH 7.4 | 150 mM NaCl | na | na |
| C3 | 7.4 | 50 mM phosphate, pH 7.4 | na | 0.001% w/v | na |
| | | | | Tween 80 | |
| C4 | 7.4 | 50 mM phosphate, pH 7.4 | na | 0.1% w/v | na |
| | | | | Tween 80 | |
| C5 | 7.4 | 50 mM phosphate, pH 7.4 | 150 mM NaCl | 0.1% Tween | na |
| | | | | 80 | |
| C6 | 7.4 | 50 mM phosphate, pH 7.4 | 16.0 mg/ml | na | 0.5 w/v % phenol |
| | | | | glycerol | |
| C7 | 7.4 | 50 mM phosphate, pH 7.4 | na | 5 w/v % | na |
| | | | | hydroxypropyl- | |
| | | | | beta- | |
| | | | | cyclodextrin | |
| C8 | 7.4 | 50 mM phosphate, pH 7.4 | na | 0.1 w/v % | na |
| | | | | human serum | |
| | | | | albumin | |
| C9 | 7.4 | 50 mM phosphate, pH 7.4 | na | 0.5 M | na |
| | | | | Sucrose | |
| C10 | 7.4 | 50 mM phosphate, pH 7.4 | na | 0.3 w/v % | na |
| | | | | Poloxamer 188 | |
| C11 | 7.4 | 50 mM phosphate, pH 7.4 | na | 18.6 mg/ml | |
| | | | | EDTA | |
| C12 | 7.7 | 50 mM phosphate, pH 7.7 | 14 mg/ml | na | 5.5 mg/ml phenol |
| | | | propylene | | |
| | | | glycol | | |
| D1 | 6.0 | 50 mM citrate, pH 6.0 | na | na | na |
| D2 | 6.0 | 50 mM citrate, pH 6.0 | 150 mM NaCl | na | na |
| D3 | 6.0 | 50 mM citrate, pH 6.0 | na | 0.001% w/v | na |
| | | | | Tween 80 | |
| D5 | 6.0 | 50 mM citrate, pH 6.0 | 150 mM NaCl | 0.1% Tween | na |
| | | | | 80 | |
| D6 | 6.0 | 50 mM citrate, pH 6.0 | 16.0 mg/ml | na | 0.5 w/v % phenol |
| | | | glycerol | | |
| D7 | 6.0 | 50 mM citrate, pH 6.0 | na | 5 w/v % | na |
| | | | | hydroxypropyl- | |
| | | | | beta- | |
| | | | | cyclodextrin | |
| D8 | 6.0 | 50 mM citrate, pH 6.0 | na | 0.1 w/v % | na |
| | | | | human serum | |
| | | | | albumin | |
| D9 | 6.0 | 50 mM citrate, pH 6.0 | na | 0.5 M | na |
| | | | | Sucrose | |
| D10 | 6.0 | 50 mM citrate, pH 6.0 | na | 0.3 w/v % | na |
| | | | | Poloxamer 188 | |
| D11 | 6.0 | 50 mM citrate, pH 6.0 | na | 18.6 mg/ml | |
| | | | | EDTA | |
| E2 | 10.0 | 50 mM glycine, pH 10.0 | 150 mM NaCl | na | na |
| E3 | 10.0 | 50 mM glycine, pH 10.0 | na | 0.001% w/v | na |
| | | | | Tween 80 | |
| E4 | 10.0 | 50 mM glycine, pH 10.0 | na | 0.1% w/v | na |
| | | | | Tween 80 | |
| E5 | 10.0 | 50 mM glycine, pH 10.0 | 150 mM NaCl | 0.1% Tween | na |
| | | | | 0.1% Tween | |
| E6 | 10.0 | 50 mM glycine, pH 10.0 | 16.0 mg/ml | na | 0.5 w/v % phenol |
| | | | | glycerol | |
| E7 | 10.0 | 50 mM glycine, pH 10.0 | na | hydroxypropyl- | na |
| | | | | beta- | |
| | | | | cyclodextrin | |
| E8 | 10.0 | 50 mM glycine, pH 10.0 | na | 0.1 w/v % | na |
| | | | | human serum | |
| | | | | albumin | |
| E9 | 10.0 | 50 mM glycine, pH 10.0 | na | 0.5 M | na |
| | | | | Sucrose | |
| E10 | 10.0 | 50 mM glycine, pH 10.0 | na | 0.3 w/v % | na |
| | | | | Poloxamer 188 | |
| F1 | 7.0 | 50 mM phosphate, pH 7.0 | na | na | na |
| F2 | 7.0 | 50 mM phosphate, pH 7.0 | 150 mM NaCl | na | na |
| F3 | 7.0 | 50 mM phosphate, pH 7.0 | na | 0.001% w/v | na |
| | | | | Tween 80 | |
| F5 | 7.0 | 50 mM phosphate, pH 7.0 | 150 mM NaCl | 0.1% Tween | na |
| | | | | 80 | |
| F6 | 7.0 | 50 mM phosphate, pH 7.0 | 16.0 mg/ml | na | 0.5 w/v % phenol |
| | | | glycerol | | |
| F7 | 7.0 | 50 mM phosphate, pH 7.0 | na | 5 w/v % | na |
| | | | | hydroxypropyl- | |
| | | | | beta- | |
| | | | | cyclodextrin | |
| F8 | 7.0 | 50 mM phosphate, pH 7.0 | na | 0.1 w/v % | na |
| | | | | human serum | |
| | | | | albumin | |
| F9 | 7.0 | 50 mM phosphate, pH 7.0 | na | 0.5 M | na |
| | | | | Sucrose | |
| F10 | 7.0 | 50 mM phosphate, pH 7.0 | na | 0.3 w/v % | na |
| | | | | Poloxamer 188 | |
| G2 | 5.0 | 50 mM citrate, pH 5.0 | 150 mM NaCl | na | na |
| G5 | 5.0 | 50 mM citrate, pH 5.0 | 150 mM NaCl | 0.1% Tween | na |
| | | | | 80 | |
| G6 | 5.0 | 50 mM citrate, pH 5.0 | 16.0 mg/ml | na | 0.5 w/v % phenol |
| | | | glycerol | | |
| G7 | 5.0 | 50 mM citrate, pH 5.0 | na | hydroxypropyl- | na |
| | | | | beta- | |
| | | | | cyclodextrin | |
| G8 | 5.0 | 50 mM citrate, pH 5.0 | na | 0.1 w/v % | na |
| | | | | human serum | |
| | | | | albumin | |
| G9 | 5.0 | 50 mM citrate, pH 5.0 | na | 0.5 M | |
| | | | | Sucrose | |
| G10 | 5.0 | 50 mM citrate, pH 5.0 | na | 0.3 w/v % | na |
| | | | | Poloxamer 188 | |
| G12 | 7.6 | 50 mM glycylglycine, pH 7.6 | na | 0.05 mg/ml | 0.21 mg/ml |
| | | | | Tween 20 | phenol |
| H2 | 3.0 | 50 mM citrate, pH 3.0 | 150 mM NaCl | na | na |
| H4 | 3.0 | 50 mM citrate, pH 3.0 na | 0.1% w/v | na | |
| | | | | Tween 80 | |
| H5 | 3.0 | 50 mM citrate, pH 3.0 | 150 mM NaCl | 0.1% Tween | na |
| 80 | | | | | |
| H6 | 3.0 | 50 mM citrate, pH 3.0 | 16.0 mg/ml | na | 0.5 w/v % phenol |
| H7 | 3.0 | 50 mM citrate, pH 3.0 | na | 5 w/v % | na |
| | | | | hydroxypropyl- | |
| | | | | beta- | |
| | | | | cyclodextrin | |
| H8 | 3.0 | 50 mM citrate, pH 3.0 na | 0.1 w/v % | na | |
| | | | | human serum | |
| | | | | albumin | |
| H9 | 3.0 | 50 mM citrate, pH 3.0 | na | 0.5 M | na |
| | | | | Sucrose | |
| H10 | 3.0 | 50 mM citrate, pH 3.0 | na | 0.3 w/v % | na |
| | | | | Poloxamer 188 | |
| H12 | 7.3 | 5.7 mM phosphate, pH 7.3 | 137 mM NaCl | 5,4 mM KCI | |

In a non-limiting embodiment, a suitable formulation that allows recovery of active FXI after freeze-drying contains:

FXI conc.: 0.2 mg/ml

Buffer: 20 mM buffer (Histidine or TRIS) (pH 5.5, 6.5 of 7.4), 25 mg/ml Mannitol (bulking agent), 2.5 mg/ml NaCl (bulking agent), with 0.01% Tween 80

Preferably, the pharmaceutical compositions are administered parenterally, i.e., intravenously, subcutaneously, or intramuscularly; intravenously being most preferred. They may also be administered by continuous or pulsatile infusion. It will be understood that any effective method for administering a FXI polypeptide may be used, including, e.g., using mucosal or inhalation methods of administration.

Local delivery of the preparations of the present invention, such as, for example, topical application, may be carried out, e.g., by means of a spray, perfusion, double balloon catheters, stent, incorporated into vascular grafts or stents, hydrogels used to coat balloon catheters, incorporation into gauze or other bandage materials, or other well established methods.

Pharmaceutical compositions of the present invention may be administered in various dosage forms, e.g. as solutions, suspensions, emulsions, microemulsions, multiple emulsion, foams, salves, pastes, plasters, ointments, tablets, coated tablets, rinses, capsules (e.g. hard gelatin capsules or soft gelatin capsules), suppositories, rectal capsules, drops, gels, sprays, powder, aerosols, inhalants, eye drops, ophthalmic ointments, ophthalmic rinses, vaginal pessaries, vaginal rings, vaginal ointments, injection solutions, in situ transforming solutions (e.g. in situ gelling, in situ setting, in situ precipitating or in situ crystallizing), infusion solution, or as implants.

Pharmaceutical compositions of the present invention may further be compounded in, or bound or conjugated to (e.g. via covalent, hydrophobic or electrostatic interactions), a drug carrier, drug delivery system or advanced drug delivery system in order to further enhance stability of the factor XI polypeptide, to increase bioavailability, to increase solubility, to decrease adverse effects, to achieve chronotherapy well known to those skilled in the art, and/or to increase patient compliance. Examples of carriers, drug delivery systems and advanced drug delivery systems include, but are not limited to, polymers, e.g. cellulose and derivatives thereof, other polysaccharides (e.g. dextran and derivatives thereof, starch and derivatives thereof), poly(vinyl alcohol), acrylate and methacrylate polymers, polylactic acid and polyglycolic acid and block co-polymers thereof, polyethyleneglycols, carrier proteins (e.g. albumin), gels (e.g. thermogelling systems, such as block co-polymeric systems well known to those skilled in the art), micelles, liposomes, microspheres, nanoparticulates, liquid crystals and dispersions thereof, L2 phase and dispersions thereof well known to those skilled in the art of phase behaviour in lipid-water systems, polymeric micelles, multiple emulsions (self-emulsifying and self-microemulsifying), cyclodextrins and derivatives thereof, and dendrimers.

Pharmaceutical compositions comprising a factor XI polypeptide prepared by use of a method according to the present invention are suitable for use in the formulation of solids, semisolids, powders and solutions for pulmonary administration using, for example, a metered dose inhaler, dry powder inhaler or a nebulizer, all of which are devices well known to those skilled in the art.

Pharmaceutical compositions comprising a factor XI polypeptide prepared by use of a method according to the present invention are suitable for use in the formulation of controlled-release, sustained-release, protracted-release, retarded-release or slow-release drug delivery systems. Pharmaceutical compositions comprising a factor XI polypeptide prepared by use of a method according to the present invention are, for instance, useful in formulation of parenteral controlled-release and sustained-release systems (both systems leading to a many-fold reduction in number of administrations) of types well known to those skilled in the art, such as controlled-release and sustained-release systems for subcutaneous administration. Without limiting the scope of the present invention, examples of useful controlled-release systems and compositions are hydrogels, oleaginous gels, liquid crystals, polymeric micelles, microspheres and nanoparticles,

Methods for producing controlled release systems useful for pharmaceutical compositions comprising a factor XI polypeptide prepared by use of a method according to the present invention include, but are not limited to, crystallization, condensation, co-crystallization, precipitation, co-precipitation, emulsification, dispersion, high-pressure homogenisation, encapsulation, spray-drying, microencapsulation, coacervation, phase separation, solvent evaporation to produce microspheres, extrusion and supercritical fluid processes. General reference is made to Handbook of Pharmaceutical Controlled Release (Wise, D. L., ed., Marcel Dekker, New York, 2000) and to Drugs and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, for example a syringe in a device of the pen type. Alternatively, parenteral administration can be performed by means of an infusion pump. A further option for administration of a composition in the form of a solution or suspension containing a factor XI polypeptide prepared by use of a method according to the present invention is administration as a nasal or pulmonary spray. As another option, pharmaceutical compositions containing a factor XI polypeptide prepared by use of a method according to the present invention may be adapted to transdermal administration, e.g. by needleless injection, by application of a patch (such as an iontophoretic patch) or by transmucosal (e.g. buccal) administration.

In one embodiment of the present invention, a pharmaceutical composition comprising a factor XI polypeptide prepared by use of a method according to the present invention is stable for more than 6 weeks of usage and for more than 3 years of storage.

In another embodiment of the present invention, a pharmaceutical composition comprising a factor XI polypeptide prepared by use of a method according to the present invention is stable for more than 4 weeks of usage and for more than 3 years of storage.

In a further embodiment of the present invention, a pharmaceutical composition comprising a factor XI polypeptide prepared by use of a method according to the present invention is stable for more than 4 weeks of usage and for more than 2 years of storage.

In an still further embodiment of the present invention, a pharmaceutical composition comprising a factor XI polypeptide prepared by use of a method according to the present invention is stable for more than 2 weeks of usage and for more than 2 years of storage.

In some embodiments, FXI polypeptide formulations have a pH from about 4.0 to about 10.0. In some embodiments, FXI polypeptide formulations have a pH from about 4.0 to about 8.0. In some embodiments, FXI polypeptide formulations have a pH from about 4.0 to about 7.0. In some embodiments, FXI polypeptide formulations have a pH from about 4.0 to about 6.5. In some embodiments, FXI polypeptide formulations have a pH from about 4.0 to about 6.0. In some embodiments, FXI polypeptide formulations have a pH of about 6.5 or below, such as, e.g., between about pH 5.0 and about 6.5; such as between about 5.5 and 6.5.

Therapeutic Administration of Factor XI:

The present invention provides for the prevention and treatment of bleeding using FXI.

Bleeding refers to extravasation of blood from any component of the circulatory system. A bleeding episode encompasses unwanted, uncontrolled and often excessive bleeding in connection with surgery, trauma, or other forms of tissue damage, as well as unwanted bleedings in subjects having bleeding disorders. Bleeding episodes may occur in subjects having a basically normal coagulation system but experiencing a (temporary) coagulophathy, as well as in subjects having congenital or acquired coagulation or bleeding disorders. In subjects having a defective platelet function, the bleedings may be likened to bleedings caused by haemophilia because the haemostatic system, as in haemophilia, lacks or has abnormal essential clotting “compounds” (e.g., platelets or von Willebrand factor protein). In subjects who experience extensive tissue damage, for example in association with surgery or vast trauma, the normal haemostatic mechanism may be overwhelmed by the demand of immediate haemostasis and they may develop excessive bleeding in spite of a basically (pre-trauma or pre-surgery) normal haemostatic mechanism. Such subjects, who further often are multi transfused, develop a (temporary) coagulopathy as a result of the bleeding and/or transfusions (i.e., a dilution of coagulation proteins, increased fibrinolysis and lowered number of platelets due to the bleeding and/or transfusions). Bleedings may also occur in organs such as the brain, inner ear region and eyes; these are areas with limited possibilities for surgical haemostasis and thus problems with achieving satisfactory haemostasis. Similar problems may arise in the process of taking biopsies from various organs (liver, lung, tumour tissue, gastrointestinal tract) as well as in laparoscopic surgery and radical retropubic prostatectomy. Common for all these situations is the difficulty in providing haemostasis by surgical techniques (sutures, clips, etc.) which also is the case when bleeding is diffuse (e.g., haemorrhagic gastritis and profuse uterine bleeding). Bleedings may also occur in subjects on anticoagulant therapy in whom a defective haemostasis has been induced by the therapy given; these bleedings are often acute and profuse. Anticoagulant therapy is often given to prevent thromboembolic disease. Such therapy may include heparin, other forms of proteoglycans, warfarin or other forms of vitamin K-antagonists, inhibitors of coagulation proteins, as well as aspirin and other platelet aggregation inhibitors, such as, e.g., antibodies or other inhibitors of GP IIb/IIIa activity. The bleeding may also be due to so-called thrombolytic therapy which comprises combined treatment with an antiplatelet agent (e.g., acetylsalicylic acid), an anticoagulant (e.g., heparin), and a fibrinolytic agent (e.g., tissue plasminogen activator, tPA). Bleeding episodes are also meant to include, without limitation, uncontrolled and excessive bleeding in connection with surgery or trauma in subjects having acute haemarthroses (bleedings in joints), chronic haemophilic arthropathy, haematomas, (e.g., muscular, retroperitoneal, sublingual and retropharyngeal), bleedings in other tissue, haematuria (bleeding from the renal tract), cerebral haemorrhage, surgery (e.g., hepatectomy), dental extraction, and gastrointestinal bleedings (e.g., UGI bleeds). The bleeding episodes may be associated with inhibitors against factor VIII; haemophilia A; haemophilia A with inhibitors; haemophilia B; deficiency of factor VII; deficiency of factor XI; thrombocytopenia; deficiency of von Willebrand factor (von Willebrand's disease); severe tissue damage; severe trauma; surgery; laparoscopic surgery; acidosis, hemodilution, consumption coagulopathies, hyperfibrinolysis, hyopthermia, haemorrhagic gastritis; taking biopsies; anticoagulant therapy; upper gastroentestinal bleedings (UGI); or stem cell transplantation. The bleeding episodes may be profuse uterine bleeding; occurring in organs with a limited possibility for mechanical haemostasis; occurring in the brain; occurring in the inner ear region; or occurring in the eyes.

A lowered count or activity of platelets refers to the number of platelets (thrombocytes) present in the subject's plasma and to the biological, coagulation-related activity of such platelets. Lowered counts may be due, e.g., to increased platelet destruction, decreased platelet production, and pooling of a larger than normal fraction of platelets in the spleen. Thrombocytopenia, for example, is defined as a platelet count less than 150,000 platelets per microliter; the upper limit of the normal platelet count is generally considered to be between 150,000 and 450,000 platelets per microliter. Platelet count may be measured by automated platelet counters; this is a well known method to the skilled worker. Syndromes due to lowered platelet count include, without limitation, thrombocytopenia, coagulophathy. Aspects of platelet activity include, without limitation, aggregation, adhesion, and coagulant activity of the platelets. Decreased activity may be due, e.g., to glycoprotein abnormalities, abnormal membrane-cytoskeleton interaction, abnormalities of platelet granules, abnormalities of platelet coagulant activity, abnormalities of signal transduction and secretion. Platelet activity, including aggregation, adhesion, and coagulant activity, are measured by standard methods known to the skilled worker, see e.g.,Platelets. A Practical Approach, Ed. S. P. Watson & K. S. Authi: Clinical Aspects of Platelet Disorders (K. J. Clemetson) 15:299-318,1996, Oxford University Press; Williams Hematology, Sixth Edition, Eds. Beutler, Lichtman, Coller, Kipps & Seligsohn, 2001, McGraw-Hill. Syndromes due to lowered platelet activity include, without limitation, Glanzmann thrombathenis, Bernard-Soulier syndrome, storage poll disease, anticoagulant treatment and thrombolytic treatment.

In the context of the present invention, treatment encompasses both prevention of bleeding, including, without limitation, prevention of an expected bleeding, such as, for example, might be expected to occur during or consequent to a surgical procedure, as well as regulation of an already occurring bleeding, such as, for example, in trauma, with the purpose of inhibiting or minimizing the bleeding. The bleeding may be at an identified site or may be at an undetermined site. Prophylactic administration of a preparation comprising a FXI polypeptide is thus included in treatment.

In some embodiments, a normal human patient, i.e., one not suffering from a congenital deficiency of FXI, may be administered FXI and/or a FXI-related polypeptide at a dosage that corresponds to about 0.05 mg to about 500 mg of wild-type FXI per day or per bleeding episode, e.g., from about 1 mg to about 200 mg, or, e.g., from about 1 mg to about 175 mg per day or per bleeding episode for a 70-kg subject as loading and maintenance doses, depending on the weight of the subject, the condition and the severity of the condition.

In some embodiments, blood is drawn from a patient in need of treatment with a FXI polypeptide and an assay is performed (prior to FXI polypeptide administration) to assess one or more of: (i) the plasma level of FXI; (ii) the ratio of activated:zymogen FXI; and/or (iii) the concentration of FXI needed to be added exogenously in order to restore effective coagulation; based on the results of the assay, an appropriate amount of FXI polypeptide is administered using a predetermined regimen. Any suitable assay may be used for these determinations, including, e.g., an ELISA or a gel-based method. Appropriate calibration standards are used in order to allow the comparison of the measured level with the usual level of FXI in human plasma (about 30 nM). Typically, it will be desired to replenish FXI levels to at least about 5 nM, preferably about 10 nM, more preferably about 15 nM, still more preferably about 20 nM, and most preferably at least about 30 nM FXI.

When a FXI-related polypeptide is being used to replenish FXI activity in a patient, the FXI-related polypeptide will exhibit a particular level of at least one FXI bioactivity and the goal of the treatment is to provide an amount of that bioactivity that corresponds to a predetermined amount of wild-type FXI (i.e., an “effective FXI plasma concentration”).

In some embodiments, the present invention encompasses therapeutic administration of FXI polypeptide to patients whose plasma level of FXI is below about 3 nM; 5 nM; or 10 nM.

Combination Treatments

The present invention also encompasses methods and compositions that provide combination therapies in which FXI polypeptide is administered with a non-Factor VII/Factor VIIa coagulation agent. Suitable non-Factor VII/Factor VIIA coagulation agents include, without limitation, Factor XII (see, e.g., WO 01/85198); inhibitors of tissue factor pathway inhibitor (TFPI inhibitors) (see, e.g., WO 01/85199); Factor IX (see, e.g., WO 02/062376); thrombin activatable fibrinolysis inhibitor (TAFI) (see, e.g., PCT/DK02/00734; PAI-1 (see, e.g., PCT/DK02/00735; Factor V (see, e.g., PCT/DK02/00736); protein C inhibitors (see, e.g., PCT/DK02/00737); thrombomodulin (see, e.g., PCT/DK02/00738); protein S inhibitors (see, e.g., PCT/DK02/00739); tissue plasminogen activator inhibitors (see, e.g., PCT/DK02/00740); a2-antiplasmin (see, e.g., PCT/DK02/00741); aprotinin (see, e.g., PCT/DK02/00742); tranexamic acid (see, e.g., PCT/DKO2/00751); E-aminocaproic acid (see, e.g., PCT/DKO2/00752); prothrombin, thrombin, Factor VII, Factor X, and fibrinogen.

The following is a list of embodiments of the present invention:

Embodiment 1: A method for treating bleeding episodes, said method comprising administering to a patient in need thereof a preparation comprising Factor XI (FXI) or FXI-related polypeptide, in an amount effective for such treatment.

Embodiment 2: A method as defined in embodiment 1, wherein said administering results in a reduced clotting time in said patient.

Embodiment 3: A method as defined in embodiment 1 or embodiment 2, wherein said administering results in an enhancement of hemostasis in said patient.

Embodiment 4: A method as defined in any of embodiments 1 to 3, wherein said administering results in an increase in clot lysis time in said patient.

Embodiment 5: A method as defined in any of embodiments 1 to 4, wherein said administering results in an increase in clot strength in said patient.

Embodiment 6: A method as defined in any of embodiments 1 to 5, wherein said administering results in an increase in overall clot quality (OCQ) in said patient.

Embodiment 7: A method as defined in any of embodiments 1 to 6, wherein, following said administration, said patient exhibits an effective FXI plasma concentration of at least about 5 nM.

Embodiment 8: A method as defined in embodiment 7, wherein said effective FXI plasma concentration is at least about 10 nM.

Embodiment 9: A method as defined in embodiment 8, wherein said effective FXI plasma concentration is at least about 30 nM.

Embodiment 10: A method as defined in any of embodiments 1 to 9, wherein said FXI or FXI-related polypeptide comprises the sequence of SEQ ID NO:1, or a fragment thereof that retains at least one FXI-associated biological activity.

Embodiment 11: A method as defined in any of embodiments 1 to 9, wherein said FXI or FXI-related polypeptide comprises the sequence of SEQ ID NO:2, or a fragment thereof that retains at least one FXI-associated biological activity.

Embodiment 12: A method as defined in any of embodiments 1 to 11, wherein said patient does not suffer from a congenital FXI deficiency.

Embodiment 13: A method as defined in any of embodiments 1 to 12, wherein said bleeding episodes are secondary to a condition selected from the group consisting of: surgery, a dental procedure, trauma, or hemodilution.

Embodiment 14: A method as defined in any of embodiments 1 to 13, further comprising, prior to said administering:

(a) obtaining a sample of blood from said patient; (b) determining at least one of: FXI concentration, ratio of FXIa:FXI, or amount of exogenous FXI necessary to restore coagulation; and (c) based on the results of step (b), determining said amount of FXI effective for treatment.

Embodiment 15: A method for treating bleeding episodes, said method comprising administering to said patient (i) a first amount of a preparation comprising a FXI polypeptide and (ii) a second amount of a preparation comprising a non-Factor VII/Factor Vila coagulation agent, wherein said first and second amounts in combination are effective for such treatment.

Embodiment 16: A method as defined in embodiment 15, wherein said non-Factor VII/Factor VIIa coagulation agent is selected from the group consisting of: Factor XIII; tissue factor pathway inhibitor (TFPI) inhibitor; Factor IX; thrombin activatable fibrinolysis inhibitor (TAFI); plasminogen activator inhibitor-i (PAI-1); Factor V; protein C inhibitor; protein S inhibitor; and tissue plasminogen activator (tPA) inhibitor.

Embodiment 17: A method as defined in embodiment 15 or embodiment 16, wherein said administering results in a reduced clotting time in said patient.

Embodiment 18: A method as defined in any of embodiments 15 to 17, wherein said administering results in an enhancement of hemostasis in said patient.

Embodiment 19: A method as defined in any of embodiments 15 to 18, wherein said administering results in an increase in clot lysis time in said patient.

Embodiment 20: A method as defined in any of embodiments 15 to 19, wherein said administering results in an increase in clot strength in said patient.

Embodiment 21: A method as defined in any of embodiments 15 to 20, wherein said administering results in an increase in overall clot quality (OCQ) in said patient.

Embodiment 22: A method as defined in any of embodiments 15 to 21, wherein, following said administration, said patient exhibits an effective FXI plasma concentration of at least about 5 nM.

Embodiment 23: A method as defined in embodiment 22, wherein said effective FXI plasma concentration is at least about 10 nM.

Embodiment 24: A method as defined in embodiment 23, wherein said effective FXI plasma concentration is at least about 30 nM.

Embodiment 25: A method as defined in any of embodiments 15 to 14, wherein said FXI or FXI-related polypeptide comprises the sequence of SEQ ID NO:1, or a fragment thereof that retains at least one FXI-associated biological activity.

Embodiment 26: A method as defined in any of embodiments 15 to 24, wherein said FXI or FXI-related polypeptide comprises the sequence of SEQ ID NO:2, or a fragment thereof that retains at least one FXI-associated biological activity.

Embodiment 27: A method as defined in any of embodiments 15 to 26, wherein said patient does not suffer from a congenital FXI deficiency.

Embodiment 28: A method as defined in any of embodiments 15 to 27, wherein said bleeding episodes are secondary to a condition selected from the group consisting of: surgery, a dental procedure, trauma, or hemodilution.

Embodiment 29: A method as defined in any of embodiments 15 to 28, further comprising, prior to said administering:

(a) obtaining a sample of blood from said patient; (b) determining at least one of: FXI concentration, ratio of FXIa:FXI, or amount of exogenous FXI necessary to restore coagulation; and (c) based on the results of step (b), determining said amount of FXI effective for treatment.

Embodiment 30: A method as defined in embodiment 1, wherein said method does not comprise administration of a Factor VII/Factor VIIa coagulation agent.

Embodiment 31: A pharmaceutical formulation comprising (i) isolated recombinant a FXI polypeptide and (ii) a pharmaceutically acceptable carrier or excipient.

Embodiment 32: Use of a FXI polypeptide for treating bleeding episodes.

Embodiment 33: Use according to embodiment 32, wherein said bleeding episodes are secondary to a condition selected from the group consisting of: surgery, a dental procedure, trauma, or hemodilution.

Embodiment 34: Use according to embodiment 32 or embodiment 33, wherein said bleeding episodes are not treated with a Factor VII/Factor VIIa coagulation agent.

Embodiment 35: Use of a FXI polypeptide for enhancement of hemostasis in a patient in need thereof .

Embodiment 36: Use of a FXI polypeptide for increasing clot lysis time in a patient in need thereof.

Embodiment 37: Use of a FXI polypeptide for increasing clot strength in a patient in need thereof.

Embodiment 38: Use of a FXI polypeptide for increasing overall clot quality (OCQ) in a patient in need thereof.

Embodiment 39: Use of a FXI polypeptide for reducing clotting time in a patient in need thereof.

Embodiment 40: Use according to any of embodiments 32 to 39, wherein the effective FXI plasma concentration in the patient is increased to at least about 5 nM.

Embodiment 41: Use according to embodiment 40, wherein the effective FXI plasma concentration is increased to at least about 10 nM.

Embodiment 42: Use according to embodiment 41, wherein the effective FXI plasma concentration is increased to at least about 30 nM.

Embodiment 43: Use according to any of embodiments 32 to 42, wherein the patient to be treated is not treated with a Factor VII/Factor VIIa coagulation agent.

Embodiment 44: Use of a FXI polypeptide for preparation of a pharmaceutical formulation for treating bleeding episodes.

Embodiment 45: Use according to embodiment 44, wherein said bleeding episodes are secondary to a condition selected from the group consisting of: surgery, a dental procedure, trauma, or hemodilution.

Embodiment 46: Use according to embodiment 44 or embodiment 45, wherein said bleeding episodes are not being treated with a Factor VII/Factor VIIa coagulation agent.

Embodiment 47: Use of a FXI polypeptide for preparation of a pharmaceutical formulation for enhancement of hemostasis in a patient in need thereof .

Embodiment 48: Use of a FXI polypeptide for preparation of a pharmaceutical formulation for increasing clot lysis time in a patient in need thereof.

Embodiment 49: Use of a FXI polypeptide for preparation of a pharmaceutical formulation for increasing clot strength in a patient in need thereof.

Embodiment 50: Use of a FXI polypeptide for preparation of a pharmaceutical formulation for increasing overall clot quality (OCQ) in a patient in need thereof.

Embodiment 51: Use of a FXI polypeptide for preparation of a pharmaceutical formulation for reducing clotting time in a patient in need thereof..

Embodiment 52: Use according to any of embodiments 47 to 51, wherein the effective FXI plasma concentration in the patient is increased to at least about 5 nM.

Embodiment 53: Use according to embodiment 52, wherein the effective FXI plasma concentration is increased to at least about 10 nM.

Embodiment 54: Use according to embodiment 53, wherein the effective FXI plasma concentration is increased to at least about 30 nM.

Embodiment 55: Use according to any of embodiments 44 to 54, wherein the patient to be treated is not treated with a Factor VII/Factor VIIa coagulation agent.

Embodiment 56: Use according to any of embodiments 32 to 55, wherein the patient to be treated does not suffer from a congenital FXI deficiency.

Embodiment 57: Use according to any of embodiments 32 to 56, wherein said FXI polypeptide comprises the sequence of SEQ ID NO:1, or a fragment thereof that retains at least one FXI-associated biological activity.

Embodiment 57: Use according to any of embodiments 32 to 56, wherein said FXI polypeptide comprises the sequence of SEQ ID NO:2, or a fragment thereof that retains at least one FXI-associated biological activity.

Embodiment 58: Use according to any of embodiments 32 to 57, wherein said FXI polypeptide is to be administered in combination with a non-Factor VII/Factor VIIa coagulation agent.

Embodiment 59: Use according to embodiment 58, wherein said non-Factor VII/Factor VIIa coagulation agent is selected from the group consisting of: Factor XII; tissue factor pathway inhibitor (TFPI) inhibitor; Factor IX; thrombin activatable fibrinolysis inhibitor (TAFI); plasminogen activator inhibitor-1 (PAI-1); Factor V; protein C inhibitor; protein S inhibitor; and tissue plasminogen activator (tPA) inhibitor.

Embodiment 60. A method for purifying a FXI polypeptide from a biological material, the method comprising subjecting the material to sequential chromatography on an cation-exchange chromatographic material, a hydrophobic interaction chromatographic material and a hydroxyapatite chromatographic material.

Embodiment 61. A method according to embodiment 60, wherein the FXI polypeptide is a recombinant FXI.

Embodiment 62. A method according to embodiment 60 or embodiment 61, wherein the FXI polypeptide is human FXI.

Embodiment 63. A method according to embodiment 60 or embodiment 61, wherein the FXI polypeptide is a dimer.

Embodiment 64. A method according to embodiment 63, wherein the FXI polypeptide is a dimer of human subunits.

Embodiment 65. A method according to any of embodiments 60 to 64, wherein the biological material is a biological fluid.

Embodiment 66. A method according to embodiment 65, wherein the biological fluid is the supernatant of a mammalian cell.

Embodiment 67. A method according to embodiment 66, wherein the biological fluid is the supernatant of a CHO culture.

Embodiment 68. A method according to any of embodiments 60 to 67, wherein the method comprises the steps of:
(a) subjecting a biological material comprising a FXI polypeptide to chromatography on a first cation-exchange chromatographic material, said chromatography comprising:
   (i) applying said biological material to said first cation-exchange chromatographic material;
   (ii) eluting unbound material from the first cation-exchange chromatographic material with a buffer A′, which buffer A is suitable for eluting material not bound to the first cation-exchange chromatographic material; and
   (iii) eluting unbound material from the first cation-exchange chromatographic material with a buffer A′, which buffer′ A is suitable for eluting material not bound to the first cation-exchange chromatographic material; and
   (iv) eluting said FXI polypeptide from the first cation-exchange chromatographic material by elution with buffer A″, which buffer A″ is suitable for eluting said FXI polypeptide from said first cation-exchange chromatographic material;
(b) subjecting the eluate from step (iv), or a fluid prepared by use of the eluate from step (iv), to chromatography using a hydrophobic interaction chromatographic material, said chromatography comprising:
   (v) applying the eluate from step (iv), or a fluid prepared by use of the eluate from step (iv), to said hydrophobic interaction chromatographic material;
   (vi) eluting unbound material from the chromatographic material with buffer B, which buffer B is suitable for eluting material not bound to the hydrophobic interaction chromatographic material; and
   (vii) eluting said FXI polypeptide from said chromatographic material by gradient-elution with buffer B′, which buffer B′ is suitable for eluting FXI from said hydrophobic interaction chromatographic material.

Embodiment 69. A method according to embodiment 68, wherein buffer A comprises one or more stabilizing agents which are capable of increasing the stability of the FXI polypeptide.

Embodiment 70. A method according to embodiment 69, wherein buffer A comprises a stabilizing agent, which stabilizing agent is a sugar, an alcohol or an alditol.

Embodiment 71. A method according to embodiment 70, wherein buffer A comprises a stabilizing agent, which stabilizing agent is a sugar, a C₄-C₈-alcohol or an alditol.

Embodiment 72. A method according to embodiment 71, wherein buffer A comprises a stabilizing agent, which stabilizing agent is a polyalcohol.

Embodiment 73. A method according to embodiment 72, wherein buffer A comprises a stabilizing agent selected from the group consisting of glycerol, propylene glycol, propan-1,3-diol, propyl alcohol and isopropyl alcohol.

Embodiment 74. A method according to embodiment 73, wherein buffer A comprises a stabilizing agent selected from the group consisting of glycerol, propylene glycol and propan-1,3-diol.

Embodiment 75. A method according to any of embodiments 72 to 74, wherein said stabilizing agent is present in a concentration of from about 5% (v/v) to about 50% (v/v).

Embodiment 76. A method according to embodiment 75, wherein said stabilizing agent is present in a concentration of from about 10% (v/v) to about 50% (v/v).

Embodiment 77. A method according to embodiment 76, wherein said stabilizing agent is present in a concentration of from about 10% (v/v) to about 20% (v/v).

Embodiment 78. A method according to embodiment 77, wherein said stabilizing agent is present in a concentration of about 10% (v/v).

Embodiment 79. A method according to embodiment 78, wherein said stabilizing agent is present in a concentration of about 20% (v/v).

Embodiment 80. A method according to any of embodiments 68 to 79, wherein the pH of buffer A is between about 6.5 and about 9.

Embodiment 81. A method according to embodiment 80, wherein the pH of buffer A is between about 7 and about 9.

Embodiment 82. A method according to embodiment 81, wherein the pH of buffer A is about 8.

Embodiment 83. A method according to any of embodiments 68 to 82, wherein buffer A has a conductivity of less than about 50 mS/cm.

Embodiment 84. A method according to any of embodiments 60 to 83, wherein the hydrophobic interaction chromatographic material uses butyl or phenyl as the ligand.

Embodiment 85. A method according to embodiment 84, wherein the hydrophobic interaction chromatographic material is Phenyl Sepharose High Performance High Substitution.

Embodiment 86. A method according to embodiment 84, wherein the hydrophobic interaction chromatographic material is Butyl Sepharose High Performance High Substitution.

Embodiment 87. A method according to any of embodiments 68 to 86, wherein the pH of buffer B is from about 6 to about 9.

Embodiment 88. A method according to embodiment 87, wherein the pH of buffer B is about 8.

Embodiment 89. A method according to any of embodiments 68 to 88, wherein buffer B has a conductivity of more than 50 mS/cm.

Embodiment 90. A method according to embodiment 89, wherein buffer B has a conductivity of more than 70 mS/cm.

Embodiment 91. A method according to any of embodiments 68 to 90, wherein the eluate from stage (vii), or a fluid prepared by use of the eluate from stage (vii), is treated by use of a method comprising a step of

(1) addition of one or more stabilizing agents which are capable of increasing the stability of the FXI polypeptide in an amount effective to significantly improve the stability thereof, and/or

(2) adjusting the pH of the eluate from stage (vii), or of a fluid prepared by use of the eluate from stage (vii), to a pH between about 7 and about 9.

Embodiment 92. A method according to embodiment 91, wherein the stabilizing agent used in step (1) is a sugar, an alcohol or an alditol.

Embodiment 93. A method according to embodiment 92, wherein the stabilizing agent used in step (1) is a sugar, a C₄-C₈-alcohol or an alditol.

Embodiment 94. A method according to embodiment 93, wherein the stabilizing agent used in step (1) is a polyalcohol.

Embodiment 95. A method according to embodiment 94, wherein the stabilizing agent used in step (1) is selected from the group consisting of glycerol, propylene glycol, propan-1,3-diol, propyl alcohol and isopropyl alcohol.

Embodiment 96. A method according to embodiment 95, wherein the stabilizing agent used in step (1) is selected from the group consisting of glycerol, propylene glycol and propan-1,3-diol.

Embodiment 97. A method according to any of embodiments 94 to 96, wherein the stabilizing agent used in step (1) is added to a concentration of from about 5% (v/v) to about 50% (v/v).

Embodiment 98. A method according to embodiment 97, wherein the stabilizing agent used in step (1) is added to a concentration of from about 10% (v/v) to about 50% (v/v).

Embodiment 99. A method according to embodiment 98, wherein the stabilizing agent used in step (1) is added to a concentration of from about 10% (v/v) to about 20% (v/v).

Embodiment 100. A method according to any of embodiments 60 to 99, wherein the method further comprises a step of subjecting the eluate from the hydrophobic interaction chromatography, or a material prepared by use of the eluate from the hydrophobic interaction chromatography, to chromatography on a Hydroxyapatite chromatographic material.

Embodiment 101. A method according to any of embodiments 68 to 100, wherein the method further comprises a step of:
subjecting the eluate from stage (vii), or a fluid prepared by use of the eluate from stage (vii), to chromatography on a hydroxyapatite chromatographic material, said chromatography comprising:
   (viii) applying the eluate (diluted and pH adjusted) from stage (vii), or a fluid prepared by use of the eluate from stage (vii), to said hydroxyapatite chromatographic material;
   (ix) eluting unbound material from the hydroxyapatite chromatographic material with buffer C, which buffer C is suitable for eluting material not bound to the hydroxyapatite chromatographic material; and
   (x) eluting said FXI polypeptide from the hydroxyapatite chromatographic material with buffer C′, wherein buffer C′ is suitable for eluting FXI polypeptides which bind to the hydroxyapatite chromatographic material in step (viii).

Embodiment 102. A method according to embodiment 101, wherein buffer C and/or buffer C′ comprises one or more stabilizing agents which are capable of increasing the stability of the FXI polypeptide.

Embodiment 103. A method according to embodiment 101, wherein a stabilizing agent is added to the fXI containing fractions, which stabilizing agent is a sugar, an alcohol or an alditol.

Embodiment 104. A method according to embodiment 103, wherein a stabilizing agent is added, which stabilizing agent is a sugar, a C₄-C₈-alcohol or an alditol.

Embodiment 105. A method according to embodiment 104, wherein a stabilizing agent is added, which stabilizing agent is a polyalcohol.

Embodiment 106. A method according to embodiment 105 wherein a stabilizing agent selected from the group consisting of glycerol, propylene glycol, propan-1,3-diol, propyl alcohol and isopropyl alcohol is added.

Embodiment 107. A method according to embodiment 106, wherein a stabilizing agent selected from the group consisting of glycerol, propylene glycol and propan-1,3-diol is added.

Embodiment 108. A method according to any of embodiments 105 to 107, wherein said stabilizing agent is added to a concentration of from about 5% (v/v) to about 50% (v/v).

Embodiment 109. A method according to embodiment 108, wherein said stabilizing agent is added to a concentration of from about 10% (v/v) to about 50% (v/v).

Embodiment 110. A method according to embodiment 109, wherein said stabilizing agent is added to a concentration of from about 10% (v/v) to about 20% (v/v).

Embodiment 111. A method according to embodiment 110, wherein said stabilizing agent is added to a concentration of about 10% (v/v).

Embodiment 112. A method according to any of embodiments 101 to 111, wherein buffer C and/or buffer C′ has a pH from about 5,8 to about 7,8.

Embodiment 113. A method according to any of embodiments 101 to 112, wherein buffer C and/or buffer C′ has a pH of about 6,0.

Embodiment 114. A pharmaceutical composition comprising a FXI polypeptide prepared by use of a method according to any of embodiments 60 to 113.

The following are intended as non-limiting examples of the present invention.

### EXAMPLES

### Example 1

### Effect of FXI on Hemostasis in Cardiac Patients

Blood was obtained before and after surgery from 5 patients undergoing cardiac surgery with cardiopulmonary bypass. The effect of FXI on clot formation and stability was evaluated using roTEG (rotational thromboelastography), using the method of Vig et al. (2001), Blood Coagulation & Fibrinolysis 12:555. Briefly, coagulation was initiated by adding Innovin (final dilution: 1:50,0000) (Dade Behring) and CaCl₂ (final concentration: 15 nM), in the presence or absence of FXI (2.5, 10, or 25 nM) (HTI/Enzyme Research Laboratories, Essen). Fibrinolysis was initiated by addition of 4 nM tPA (American Diagnostica). Measurements were made using a ROTEG-04 Whole Blood Haemostasis System Rotation Thrombelastography apparatus (Pentapharm GmBH). Overall Clot Quality (OCQ) is calculated as:
Max vel/t_{max vel})×(t_{min vel}−t_{max vel})

OCQ is then normalized to the control sample (incubated in the absence of any hemostatic agents.

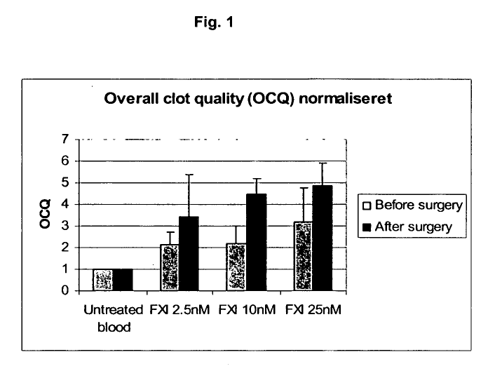

The results are shown in FIG. 1. FXI considerably improved the overall clot formation and clots formed in the presence of FXI had an increased resistant to fibrinolysis.

### Example 2

### Effect of FXI on Hemostasis in Normal Blood

Blood was obtained from 4 normal subjects, and the effect of FXI on clot formation was evaluated by ROTEG as described in Example 1.

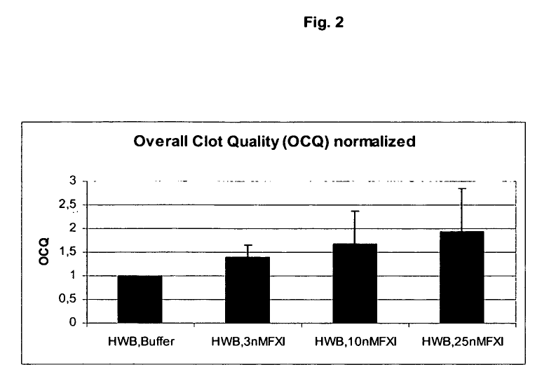

FIG. 2 illustrates that FXI caused a dose-dependent increase is OCQ in normal blood.

### Example 3

### Activity of Glycosylation-disrupted FXI Polypeptides

FXI variant containing the following substitutions were constructed using standard methodologies and were expressed after transfection in HEK293 cells. Crude cell culture supernatants were collected from cells grown for 96 h at 37° C. FXI activity was measured by ROTEG as described in Example 1.

The results are shown in the following Table.

| | FXI activity in % of expected |
|---|---|
| Protein | values |
| NHP (Normal human plasma) (31 nM | 106 |
| FXI) | |
| FXI N72Q - 1.2 nM | 42 |
| FXI N108Q - 1.3 nM | 62 |
| FXI N335Q - 0.4 nM | 75 |
| FXI N432Q - 1.2 nM | 33 |
| FXI N473Q - 0.6 nM | 83 |

### Example 4

### Storage Stability of FXI Formulations

The following solutions of FXI were prepared and stored for 5 weeks at 5° C., after which FXI activity was measured as described in Example 1.
1. 384 nM FXI in 4 mM acetate, 150 mM NaCl, pH 5.4
2. 190 nM FXI in 50 mM acetate buffer, 150 mM NaCl, pH 5.4
3. 190 nM FXI in 50 mM acetate buffer, 150 mM NaCl, pH 5.4, 1 mM CaCl2
4. 190 nM FXI in 50 mM acetate buffer, 75 mM NaCl, pH 5.4, 300 mg/ml sucrose
5. 190 nM FXI in 50 mM MES buffer, pH 6.5, 150 mM NaCl
6. 190 nM FXI in 50 mM MES buffer, pH 6.5, 150 mM NaCl, 1 mM CaCl2
7. 190 nM FXI in 50 mM MES buffer, pH 6.5, 75 mM NaCl, 300 mg/ml sucrose

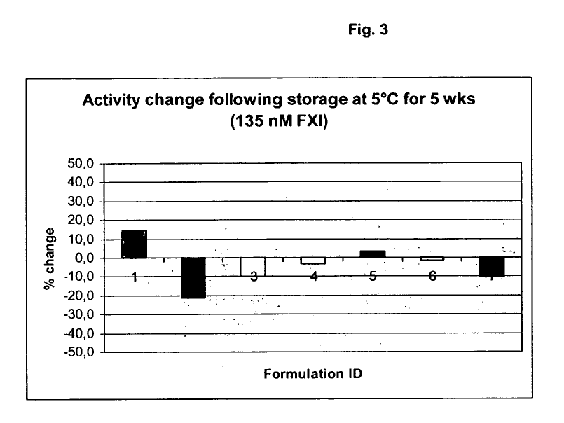

The results are shown in FIG. 3.

### Example 5

### Binding Peptides for FXI

The following experiments were performed to identify peptides that bind FXI.

1. Synthesis of Peptide Libraries:

The following libraries were synthesized using Fmoc solid phase peptide synthesis on Tentagel resin bead from Rapp Polymere (Germany). Three different peptide bead libraries were used in the screening. They are named BL 121, BL 122 and BL 123.

The format of the library BL 121 is:

O₁-O₂-O₃-O₄-O₅-O₆-O₇-O₈-O₉-O₁₀-O₁₁-O₁₂-O₁₃-O₁₄-Tentagel resin, where On is a L-amino acid and n=1,2 and 11,12 can be any proteinogenic L-amino acid except methionine and cysteine and n=4,5 and 7,8 and 10,11 and 13,14 can be any proteinogenic L-amino acid except Methionine and cysteine and deletion, and n=3,6,9,12 can be Phe, Trp, Tyr, Leu.
   The format of the library BL 122 is
O₁-O₂-O₃-O₄-O₅-O₆-O₇-O₈-O₉-O₁₀-O₁₁-O₁₂-Tentagel resin, where On is a L-amino acid and n=1-12 can be any proteinogenic L-amino acid except methionine and cysteine.
   The format of the library BL 124 is:
O₁-O₂-O₃-O₄-O₅-O₆-O₇-Asp-Phe-Pro -O₈-O₉-O₁₀-O₁₁-Tentagel resin, where On is a L-amino acid and n=1-11 can be any proteinogenic L-amino acid except methionine and cysteine.
   2. Screening the Peptide Bead Libraries:

Recombinant factor XI from Heamatologic Technologies was purchased and biotinylated according to standard laboratory protocols. Then 5 ul of factor XI (1.2 uM) and 1 ul streptavidin-alkaline phosphatase (1 mg/ml, Sigma) were added to three synthetic peptide bead libraries, BL 121, BL 122 and BL 124, respectively, and allowed to incubate for about 2-3 hours. The incubation buffer was 15 mM TRIS-HCl, pH=7.4, 0.15M NaCl, 0.5% bovine Serume Albumin (BSA) and 0.05% Tween20. After washing with washing buffer (M TRIS-HCl, pH=7.4, 0.15M NaCl, and 0.05% Tween20), BCIP and NBT were added in colorbuffer (50 mM TRIS-HVI pH=8.8, 0.15M NaCl, 0.05% Tween20 and 15 mM MgCl2) and coloration was allowed to proceed 30 min-1.5 h.

3. Sequence Determination

Active blue beads were removed from the library and sequenced by the Edman sequencer (Procise, Applied Biosystems).

Results:

Specific factor XI and factor XI-like binding peptides found in library BL 121, BL 122 and BL 123 according to the invention include peptides comprising amino acid sequences are outlined below:

Sequences found in BL121

| | |
|---|---|
| SEQ ID NO:03: | SRWPWSVFPDFPD |
| SEQ ID NO:04: | DVWDYVVFDDFPS |
| SEQ ID NO:05: | QRWVPYDDFPSLRS |
| SEQ ID NO:06: | RHFHVFPDFPFVH |
| SEQ ID NO:07: | HHFPPFSHFPDLPQ |
| SEQ ID NO:08: | RRLPLSRLPDFP |
| SEQ ID NO:09: | HPFFRGYPDFPD |
| SEQ ID NO:10: | HPWHLVYPDFPS |
| SEQ ID NO:11: | HDWLVRWPDFPS |
| SEQ ID NO:12: | SHFWRQWPDFSD |
| SEQ ID NO:13: | PQLRWHDFPDFGS |
| SEQ ID NO:14: | VVWRHWQDFDQFW |
| SEQ ID NO:15: | VDWQWSRFDDFPS |
| SEQ ID NO:16: | HPWFDDFPHLFQ |

Sequences from library BL122

| | |
|---|---|
| SEQ ID NO:17: | YKWIHHDDFPLV |
| SEQ ID NO:18: | FDRKRVHPDFPH |
| SEQ ID NO:19: | DVWDYVVFDDFPS |
| SEQ ID NO:20: | QQPIQRFPDFP |
| SEQ ID NO:21: | QAIFTRFPDFPN |
| SEQ ID NO:22: | EWFPDFPEGSDG |
| SEQ ID NO:23: | HTHAFPDFPPH |
| SEQ ID NO:24: | LVKGFPDFPNHN |
| SEQ ID NO:25: | GPFPYAYEDFPE |
| SEQ ID NO:26: | FYLKTRYYDFPE |
| SEQ ID NO:27: | FQARHTIGDFPA |
| SEQ ID NO:28: | RIKDFPSDSNTV |
| SEQ ID NO:29: | IWESHKVIEDFP |
| SEQ ID NO:30: | QWFSVSRYQDFD |
| SEQ ID NO:31: | QKDFHWRILPDF |
| SEQ ID NO:32: | KIVKFPHTFPDL |
| SEQ ID NO:33: | HLYDFDLDNEY |
| SEQ ID NO:34: | KTILGDVDFDI |
| SEQ ID NO:35: | RQLHPFHHFHG |
| SEQ ID NO:36: | RSWLRYGYGH |
| SEQ ID NO:37: | FNWNNVDEYYDW |
| SEQ ID NO:38: | DQWDWEDYDEAW |
| SEQ ID NO:39: | YDIYDDYEIWA |

Sequences found in BL124

| | |
|---|---|
| SEQ ID NO:40: | YPKHIYADFPSTRL |
| SEQ ID NO:41: | YPRHIYPDFPTDTT |
| SEQ ID NO:42: | YLKHAWPDFPKLQQ |
| SEQ ID NO:43: | YVRHRFEDFPTALP |
| SEQ ID NO:44: | FPWHKYEDFPSPRT |
| SEQ ID NO:45: | QPAHRYPDFPRNNH |
| SEQ ID NQ:46: | LPKTRFLDFPHVSF |
| SEQ ID NO:47: | LPPARYPDFPAAKK |
| SEQ ID NO:48: | IPKNRFSDFPDAQG |
| SEQ ID NO:49: | LPSFRFPDFPATKT |
| SEQ ID NO:50: | RVLNRYPDFPTTNQ |
| SEQ ID NO:51: | FFKKTYADFPTSQT |
| SEQ ID NO:52: | IFKKTYEDFPRFVY |
| SEQ ID NO:53: | VLHNKYDDFPRVKK |
| SEQ ID NO:54: | KVKHRFNDFPVWGN |

It is concluded that useful FXI-binding peptides include those having a core amino acid motif of Asp-Phe-Pro.

### Example 6

The cells from mammalian cell culture was separated from the supernatant by centrifugation or filtration. Benzamidine and EDTA was added to final concentration 1 mM.

### Example 7

First Cation-exchange Chromatography Using Obelix ST CIEX (cat no 11-0010)

A Obelix matrix was equilibrated with 5 column volumes (cv) of buffer A, and a load corresponding to 150 ml supernatant pr ml packed column was applied to the column. The column was washed with 4 cv of buffer A (30 mM Tris pH 8,0) and then with 5 cv of buffer A′ (50 mM Tris 50% Glycerol_{87%} pH 9,0) . Elution was then performed with 5 cv buffer A″ (50 mM Tris 50% Glycerol_{87%} 1M NaCl pH 9,0). Flowrate was 16 cv/h, temperature was 0-10° C. The column was regenerated with 1M NaOH. Fractions were collected from at about 50% of peak height, the first peak eluting is discarded but the next main peak contains FXI. Analysis of FXI polypeptide-containing fractions was performed by HPLC (vide infra) using C4 Jupiter Phenomonex cat no OOG-4167-EO, 4.6×250 mm and by SDS-PAGE on a NUpage 4-12% Bis/Tris Gel (Invitrogen) with MOPS running buffer under reductive conditions. Benzamidine and EDTA were added (to 1 mM) to the fractions containing FXI polypeptide and kept at approx. 4° C. in a refrigerator, or frozen at −80° C., until further use.

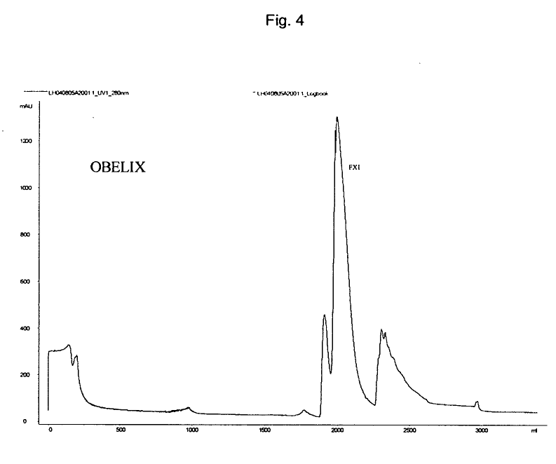

Alternative to Obelix ST CIEX is Streamline Direct CST Amersham cat no 17-5266-03. (FIG. 4, preparative chromatograme)

### Example 8

Hydrophobic Interaction Chromatography Using Butyl Sepharose High Performance High Substitution (cat no 17-3100)

1,5 volume of a buffer containing 2M NaCl 40 mM Tris pH 8 was added to the combined fractions containing FXI polypeptide from Example 7, and the pH was adjusted to 8,2 if not already between 8,0 and 8,4. A Butyl Sepharose High Performance High Substitution matrix was equilibrated with 3 cv of buffer B (1M NaCl 20 mM Tris pH 8,0) and a load corresponding to approximately 1 mg/ml was applied to the column. The column was then washed with 2 cv of buffer B and then subjected to gradient elution going from buffer B to 100% elution buffer B′ (20 mM Tris pH 8,0) over 20 cv followed by 2 cv of 100% elution buffer B′. Flow rate was 12 cv/h, temperature was 0-10° C. Fractions were collected after elution of approximately 10 cv and until 15 cv. Analysis of FXI polypeptide-containing fractions was performed by HPLC (vide infra) using C4 Jupiter Phenomonex cat no OOG-4167-EO, 4.6×250 mm and by SDS-PAGE on a NUpage 4-12% Bis/Tris Gel (Invitrogen) with MOPS running buffer under reductive conditions. A 1/14 volume of propylene glycol was immediately added to the pool of FXI polypeptide-containing fractions to a final concentration of 20% (v/v) propylene glycol, and the resulting pool was then kept at approx. 4° C., or frozen, until further use

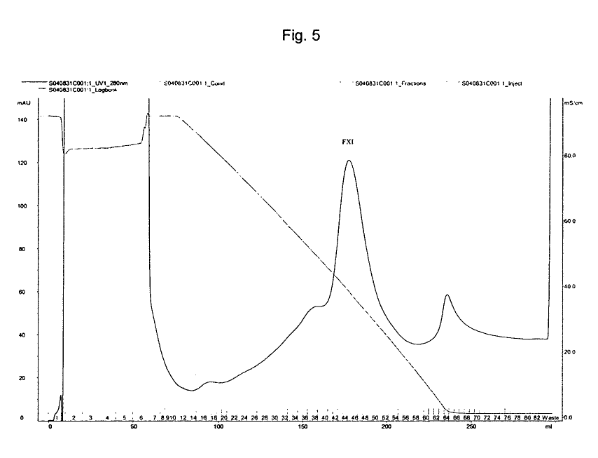

Alternative to Butyl Sepharose High Performance High Substitution is Phenyl Sepharose High Performance High Substitution. This alternative matrix results in later elution. (FIG. 5, preparative chromatograme)

### Example 9

Hydroxyapatite Chromatography Using CHT Hydroxvapatite Type I BioRad cat no 157-0020)

The pH of the pool of FXI polypeptide-containing fractions from Example 8 was adjusted to 6,0 and 1 volume water was added to a conductivity of below 20 mS/cm. A Hydroxyapatite Type I 20 μm matrix was equilibrated with 6 cv of buffer C, and then a load corresponding to 5 mg/ml gel was applied to the column. The column was then washed with 15 cv of buffer C′ (20 mM K-PO4 pH 6,0), and a buffer containing 95% buffer C and 5% elution buffer C′ (20 mM K-PO4 2M NaCl pH 6,0) was performed as a washing step. A gradient elution from 5% C′ to 100% C′ was performed and used to elute the FXI polypeptide in small fractions. The conductivity of the pool containing the FXI polypeptide fractions was about 60 mS/cm and the pH about 6,0. Analysis of FXI polypeptide-containing fractions was performed by HPLC (vide infra) using C4 Jupiter Phenomonex cat no OOG-4167-EO, 4.6×250 mm and by SDS-PAGE on a NUpage 4-12% Bis/Tris Gel (Invitrogen) with MOPS running buffer under reductive conditions. The HPLC purity is >97% and concentration of FXI is about 1,2 mg/ml.

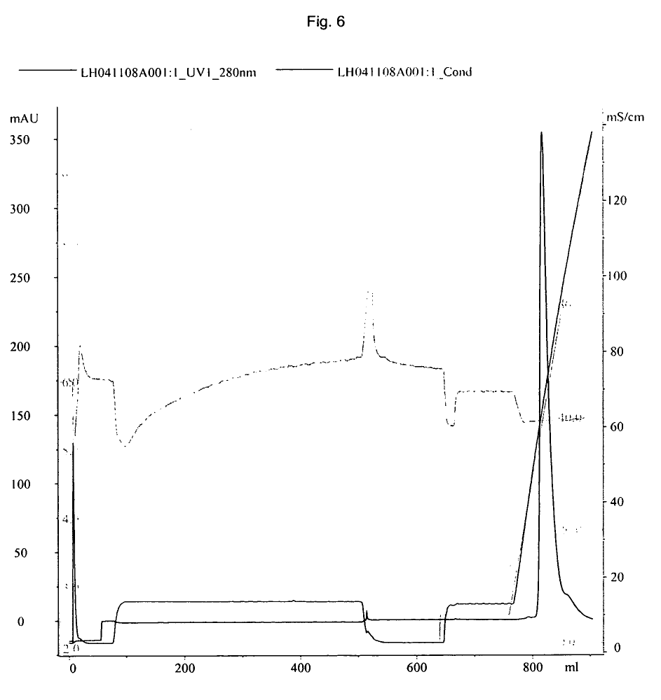

The FXI containing fraction of high purity was collected and Propylene Glycol was added to final 10% v/v and stored below −20° C. (FIG. 6, preparative chromatograme)

### Example 10

HPLC Analysis Procedure

High-Performance Liquid Chromatography (HPLC; referred to in Examples 7-9, above) was performed using C4 Jupiter Phenomonex cat no OOG-4167-EO, 4.6×250 mm and employing buffers as follows:
Buffer I: 0.1% TFA in H₂O
Buffer II: 0.07% TFA in CH₃CN

Equilibration of the column was carried out using a mixture of 75% (v/v) Buffer I with 25% (v/v) Buffer II for 5 minutes (flow rate 1 ml/min.).

Elution of the column took place using a gradient going from 75% Buffer I/25% Buffer II to 39% Buffer I/61% Buffer II over a period of 18 minutes (flow rate 1 ml/min.).

Regeneration of the column was performed by washing with 100% Buffer II for 2 minutes. (flow rate 0.5 ml/min.). The detection wavelength employed was 214 nm. Temperature was 50° C. Samples of from 2 to 50 μg were loaded onto the column.

All patents, patent applications, and literature references referred to herein are hereby incorporated by reference in their entirety.

Many variations of the present invention will suggest themselves to those skilled in the art in light of the above detailed description. Such obvious variations are within the full intended scope of the appended claims.

1. A method for treating bleeding episodes, said method comprising administering to a patient in need thereof a preparation comprising an isolated Factor XI (FXI) or FXI-related polypeptide, in an amount effective for such treatment.

2. A method as defined in claim 1, wherein said administering results in a reduced clotting time in said patient.

3. A method as defined in claim 1, wherein said administering results in an enhancement of hemostasis in said patient.

4. A method as defined in claim 1, wherein said administering results in an increase in clot lysis time in said patient.

5. A method as defined in claim 1, wherein said administering results in an increase in clot strength in said patient.

6. A method as defined in claim 1, wherein said administering results in an increase in overall clot quality (OCQ) in said patient.

7. A method as defined in claim 1, wherein, following said administration, said patient exhibits an effective FXI plasma concentration of at least about 5 nM.

8. A method as defined in claim 7, wherein said effective FXI plasma concentration is at least about 10 nM.

9. A method as defined in claim 8, wherein said effective FXI plasma concentration is at least about 30 nM.

10. A method as defined in claim 1, wherein said FXI or FXI-related polypeptide comprises the sequence of SEQ ID NO:1, or a fragment thereof that retains at least one FXI-associated biological activity.

11. A method as defined in claim 1, wherein said FXI or FXI-related polypeptide comprises the sequence of SEQ ID NO:2, or a fragment thereof that retains at least one FXI-associated biological activity.

12. A method as defined in claim 1, wherein said patient does not suffer from a congenital FXI deficiency.

13. A method as defined in claim 1, wherein said bleeding episodes are secondary to a condition selected from the group consisting of: surgery, a dental procedure, trauma, or hemodilution.

14. A method as defined in claim 1, further comprising, prior to said administering:
(a) obtaining a sample of blood from said patient; (b) determining at least one of: FXI concentration, ratio of FXIa:FXI, or amount of exogenous FXI necessary to restore coagulation; and (c) based on the results of step (b), determining said amount of FXI effective for treatment.

15. A method for treating bleeding episodes, said method comprising administering to said patient (i) a first amount of a preparation comprising a FXI polypeptide and (ii) a second amount of a preparation comprising a non-Factor VII/Factor VIIa coagulation agent, wherein said first and second amounts in combination are effective for such treatment.

16. A method as defined in claim 15, wherein said non-Factor VII/Factor VIIa coagulation agent is selected from the group consisting of: Factor XIII; tissue factor pathway inhibitor (TFPI) inhibitor; Factor IX; thrombin activatable fibrinolysis inhibitor (TAFI); plasminogen activator inhibitor-1 (PAI-1); Factor V; protein C inhibitor; protein S inhibitor; and tissue plasminogen activator (tPA) inhibitor.

17. A method as defined in claim 1, wherein said method does not comprise administration of a Factor VII/Factor VIIa coagulation agent.

18. A pharmaceutical formulation comprising (i) an isolated recombinant FXI polypeptide and (ii) a pharmaceutically acceptable carrier or excipient.

19. A method as defined in claim 1, wherein said FXI or FXI-related polypeptide is isolated from a recombinant source.
